SEQUENCE LISTING (33 sequences)

SEQ ID NO: 1 - protein, 106 aa, Mus musculus
QSPASLAVSLGQRATISCRASESVDSFGNSFMHWYQQKAGQPPKSSIYRAANLESGIPARFSGSGSRTDFTLTINPVEADDVATYFCQQSYEDPWTFGGGTKLGNQ

SEQ ID NO: 2 - protein, 105 aa, Mus musculus
LVKPGGSLKLSCVASGFTFSSYAMSWVRQTPEKRLEWVASISSGGTTYYPDNVKGRFTISRDNARNILYLQMSSLRSEDTAMYYCARGYYDYHYWGQGTTLTVSS

SEQ ID NO: 3 - protein, 106 aa, Mus musculus
QSPASLAVSLGQRATISCRASESVDSYVNSFLHWYQQKPGQPPKLLIYRASNLQSGIPARFSGSGSRTDFTLTINPVEADDVATYCCQQSNEDPTTFGGGTKLEIK

SEQ ID NO: 4 - protein, 112 aa, Mus musculus
LQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSA

SEQ ID NO: 5 - protein, 101 aa, Mus musculus
QTPSSLSASLGDRVTISCRASQDIKNYLNWYQQPGGTVKVLIYYTSRLHSGVPSRFSGSGSGTDYSLTISNLEQEDFATYFCQRGDTLPWTFGGGTKLEIK

SEQ ID NO: 6 - protein, 116 aa, Mus musculus
LESGPGLVAPSQSLSITCTVSGFSLTNYDISWIRQPPGKGLEWLGVIWTSGGTNYNSAFMSRLSITKDNSESQVFLKMNGLQTDDTGIYYCVRGDGNFYLYNFDYWGQGTTLTVSS

SEQ ID NO: 7 - protein, 15 aa, Mus musculus
RASESVDSFGNSFMH

SEQ ID NO: 8 - protein, 7 aa, Mus musculus
RAANLES

SEQ ID NO: 9 - protein, 9 aa, Mus musculus
QQSYEDPWT

SEQ ID NO: 10 - protein, 5 aa, Mus musculus
SYAMS

SEQ ID NO: 11 - protein, 16 aa, Mus musculus
SISSGGTTYYPDNVKG

SEQ ID NO: 12 - protein, 7 aa, Mus musculus
GYYDYHY

SEQ ID NO: 13 - protein, 15 aa, Mus musculus
RASESVDSYVNSFLH

SEQ ID NO: 14 - protein, 7 aa, Mus musculus
RASNLQS

SEQ ID NO: 15 - protein, 9 aa, Mus musculus
QQSNEDPTT

SEQ ID NO: 16 - protein, 6 aa, Mus musculus
SDSAWN

SEQ ID NO: 17 - protein, 16 aa, Mus musculus
YISYSGSTSYNPSLKS

SEQ ID NO: 18 - protein, 6 aa, Mus musculus
GLRFAY

SEQ ID NO: 19 - protein, 11 aa, Mus musculus
RASQDIKNYLN

SEQ ID NO: 20 - protein, 7 aa, Mus musculus
YTSRLHS

SEQ ID NO: 21 - protein, 9 aa, Mus musculus
QRGDTLPWT

SEQ ID NO: 22 - protein, 5 aa, Mus musculus
NYDIS

SEQ ID NO: 23 - protein, 16 aa, Mus musculus
VIWTSGGTNYNSAFMS

SEQ ID NO: 24 - protein, 12 aa, Mus musculus
GDGNFYLYNFDY

SEQ ID NO: 25 - protein, 106 aa, Artificial Sequence
  IC14 VL (short)
QSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIK

SEQ ID NO: 26 - protein, 112 aa, Artificial Sequence
  IC14 VH (short)
LQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSS

SEQ ID NO: 27 - protein, 9 aa, Artificial Sequence
  IC14 L-CDR3
QQSNEDPYT

SEQ ID NO: 28 - protein, 213 aa, Artificial Sequence
  IC14 light chain (short)
QSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 439 aa, Artificial Sequence
  IC14 heavy chain (short)
LQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 111 aa, Artificial Sequence
  IC14 VL (full)
DIVLTQSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIK

SEQ ID NO: 31 - protein, 115 aa, Artificial Sequence
  IC14 VH (full)
DVQLQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSS

SEQ ID NO: 32 - protein, 218 aa, Artificial Sequence
  IC14 light chain (full)
DIVLTQSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 442 aa, Artificial Sequence
  IC14 heavy chain (full)
DVQLQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0276059 A1
(43) Pub. Date: Sep. 4, 2025

(54) THERAPEUTIC METHODS AND AGENTS FOR THE TREATMENT OF MYOCARDIAL INFARCTION

(71) Applicant: Implicit Bioscience Limited, Woolloongabba (AU)

(72) Inventors: Brian ZIEGELAAR, Wakerley (AU); Garry REDLICH, East Brisbane (AU)

(73) Assignee: Implicit Bioscience Limited, Woolloongabba (AU)

(21) Appl. No.: 18/024,738

(22) PCT Filed: Sep. 10, 2021
(86) PCT No.: PCT/AU2021/051049

§ 371 Date: Mar. 3, 2023

(30) Foreign Application Priority Data
Sep. 10, 2020 (AU) 2020903245

(51) Int. Cl. A61K 39/395; A61K 39/00; A61K 45/06; A61P 9/10; C07K 16/28

(52) U.S. Cl. CPC A61K 39/3955; A61K 45/06; A61P 9/10; C07K 16/2896; A61K 2039/505; A61K 2039/545; C07K 2317/54; C07K 2317/76

(57) ABSTRACT

This disclosure relates generally to methods and agents for treating myocardial infarction. More particularly, the present disclosure relates to the use of CD14 antagonist antibodies for treating myocardial infarction.

### CROSS-REFERENCE TO RELATED APPLICATIONS

This application is a national stage application under 35 U.S.C. § 371 of International Application No. PCT/AU2021/051049, filed internationally on Sep. 10, 2021, which claims priority to Australian Provisional Application No. 2020903245 entitled “Therapeutic methods and agents for the treatment of myocardial infarction” filed 10 Sep. 2020, the contents of which are incorporated herein by reference in their entirety.

### INCORPORATION BY REFERENCE OF SEQUENCE LISTING

The present application is being filed along with a Sequence Listing in electronic format. The Sequence Listing is provided as a file entitled 229752009800SeqList.txt, created Mar. 3, 2023, which is 25,187 bytes in size. The information in the electronic format of the Sequence Listing is incorporated by reference in its entirety.

### FIELD OF THE INVENTION

This disclosure relates generally to methods and agents for treating myocardial infarction. More particularly, the present disclosure relates to the use of CD14 antagonist antibodies for treating myocardial infarction.

### BACKGROUND OF THE INVENTION

Heart disease and in particular myocardial infarction (MI) is a significant cause of mortality and morbidity across the world. For example, in the US, about 1 million myocardial infarctions occur annually, resulting in death for around 300,000 to 400,000 people. For those who survive MI, long-term heart damage can result, reducing life expectancy and quality of life.

MI refers to tissue death (i.e. infarction) of the heart muscle, or the myocardium, as a result of ischaemia. Myocardial infarction occurs when the blood supply to the heart does not meet the oxygen demand of the muscle. This is typically the result of an occlusion (or blockage) of a coronary artery, such as following the rupture of vulnerable atherosclerotic plaque and the formation of a blot clot. Other less-common causes include coronary artery embolism, cocaine-induced ischemia, coronary dissection, and coronary vasospasm.

MI can be divided into two categories for the purposes of treatment: non-ST-segment elevation MI (NSTEMI) and ST-segment elevation MI (STEMI). STEMI most commonly occurs when thrombus formation results in complete occlusion of a major epicardial coronary vessel, and is the most serious form, being a life-threatening, time-sensitive emergency that must be diagnosed and treated promptly. For STEMI, emergency reperfusion is achieved by percutaneous coronary intervention (PCI, e.g. angioplasty or stent placement), fibrinolytic drugs (e.g. streptokinase, anistreplase or a tissue plasminogen activator (tPA; e.g. tenecteplase, reteplase or alteplase)), or, occasionally, coronary artery bypass graft surgery. For NSTEMI, reperfusion is via percutaneous intervention or coronary artery bypass graft surgery; fibrinolytic therapies are not used in NSTEMI. All MI patients are typically treated with beta blockers, high intensity statins, angiotensin converting enzyme (ACE) inhibitors and/or platelet inhibitors (e.g. aspirin and/or P2Y12 inhibitors such as ticlopidine, clopidogrel, ticagrelor and prasugrel).

Despite these interventions, many patients with MI have permanent damage to the heart. Myocardial injury leads to activation of a stereotyped inflammatory cascade, comprised of early neutrophil ingress followed by monocyte/macrophage infiltration. Between days 3-5 following MI there is a transition from inflammation to repair, with activation of fibroblasts and progressive scar deposition. Over time there is pathological remodelling, with changes to the ventricular geometry, wall thinning, ischaemic mitral regurgitation and further cardiomyocyte loss. The development of scar tissue and ventricular remodelling following MI puts the patient at risk for potentially life threatening arrhythmias and heart failure. Indeed, at least 5%-10% of survivors of MI die within the first 12 months after the MI, and close to 50% need hospitalization within the same year. The overall prognosis of a patient suffering MI depends on the extent of muscle damage following MI and the adverse remodeling associated with that damage. Although better outcomes are seen in patients who undergo early PCI or fibrinolytic therapy, there remains a need for additional agents and methods for treating MI so as to further reduce muscle damage, detrimental fibrosis and improve outcomes.

### SUMMARY OF THE INVENTION

The present invention arises in part from the surprising determination that targeting Cluster of Differentiation 14 (CD14), such as by administration of an anti-CD14 antagonist antibody, can reduce or ameliorate the cardiac damage resulting from MI. In particular, it has been demonstrated for the first time herein that administration of a CD14 antagonist antibody following STEMI, the most severe form of MI, improved systolic function, contractile properties and heamodynamic function of the heart (e.g. increased stroke volume, cardiac output, ejection fraction, stroke work and dV/dt max, and reduced dV/dt min) and decreased infarct size and reduced fibrosis compared to when the antibody was not administered. This clear improvement in multiple MI parameters, representing a significant improvement in cardiac efficiency and function, is surprising for any one agent and particularly for an anti-CD14 agent given previous findings that suggested that targeting CD14 would have no effect on preventing or ameliorating the deleterious consequences of MI infarct size or contractile properties (see e.g. Arslan et al. Impact of CD14 deficiency on ischemia reperfusion injury, Immunotherapy@Brisbane 2017, Brisbane, Australia, 10-12 May 2017).

Damage-Associated Molecular Pattern (DAMP) molecules are released by damaged cardiomyocytes during MI and cause resident pro-inflammatory macrophages to attract circulating leukocytes (primarily neutrophils) from the blood. Following phagocytosis of the damaged and necrotic cells, these neutrophils undergo apoptosis, which promotes the resolution-phase of tissue repair. CD14 is an important cofactor for a number of pattern recognition receptors which promote DAMP-driven inflammation in a variety of cell types, including circulating and infiltrating monocytes and macrophages. Without being bound by theory, it is proposed that targeting CD14 reduces the excessive inflammation associated with MI and mitigates subsequent damage, fibrosis and remodelling in the heart. In some embodiments of the present disclosure, CD14 is targeted only in the acute phase (i.e. up to 72-96 hours post MI). Without being bound by theory, it is proposed that doing so targets and reduces the effect of pro-inflammatory “M1” monocytes/macrophages during this phase, while allowing the reparative, anti-inflammatory “M2” monocytes/macrophages to function in tissue repair in the later phase.

Thus, in one aspect, provided is a method for treating myocardial infarction (MI) in a subject, comprising, consisting or consisting essentially of administering an effective amount of a CD14 antagonist antibody to the subject. In another aspect, provided is use of a CD14 antagonist antibody for the preparation of a medicament for the treatment of MI.

In some embodiments, the CD14 antagonist antibody is administered to the subject up to 72 hours post-MI or post-MI diagnosis (e.g. up to 12, 18, 24, 36 or 48 hours post-MI or post-MI diagnosis). In some examples, the CD14 antagonist antibody is administered to the subject in 1, 2, 3 or more doses. In one embodiment, the CD14 antagonist antibody is administered systemically.

In some embodiments, the MI is ST-segment elevation MI (STEMI). In other embodiments, the MI is non-ST-segment elevation MI (NSTEMI).

In one example, the CD14 antagonist antibody is selected from:
(i) an antibody that comprises: a) an antibody VL domain, or antigen binding fragment thereof, comprising L-CDR1, L-CDR2 and L-CDR3, wherein: L-CDR1 comprises the sequence RASESVDSFGNSFMH [SEQ ID NO: 7](3C10 L-CDR1); L-CDR2 comprises the sequence RAANLES [SEQ ID NO: 8](3C10 L-CDR2); and L-CDR3 comprises the sequence QQSYEDPWT [SEQ ID NO: 9](3C10 L-CDR3); and b) an antibody VH domain, or antigen binding fragment thereof, comprising H-CDR1, H-CDR2 and H-CDR3, wherein: H-CDR1 comprises the sequence SYAMS [SEQ ID NO: 10](3C10 H-CDR1); H-CDR2 comprises the sequence SISSGGTTYYPDNVKG [SEQ ID NO: 11](3C10 H-CDR2); and H-CDR3 comprises the sequence GYYDYHY [SEQ ID NO: 12](3C10 H-CDR3);
(ii) an antibody that comprises: a) an antibody VL domain, or antigen binding fragment thereof, comprising L-CDR1, L-CDR2 and L-CDR3, wherein: L-CDR1 comprises the sequence RASESVDSYVNSFLH [SEQ ID NO: 13](28C5 L-CDR1); L-CDR2 comprises the sequence RASNLQS [SEQ ID NO: 14](28C5 L-CDR2); and L-CDR3 comprises the sequence QQSNEDPTT [SEQ ID NO: 15](28C5 L-CDR3); and b) an antibody VH domain, or antigen binding fragment thereof, comprising H-CDR1, H-CDR2 and H-CDR3, wherein: H-CDR1 comprises the sequence SDSAWN [SEQ ID NO: 16](28C5 H-CDR1); H-CDR2 comprises the sequence YISYSGSTSYNPSLKS [SEQ ID NO: 17](28C5 H-CDR2); and H-CDR3 comprises the sequence GLRFAY [SEQ ID NO: 18](28C5 H-CDR3);
(iii) an antibody that comprises: a) an antibody VL domain, or antigen binding fragment thereof, comprising L-CDR1, L-CDR2 and L-CDR3, wherein: L-CDR1 comprises the sequence RASESVDSYVNSFLH [SEQ ID NO: 13](IC14 L-CDR1); L-CDR2 comprises the sequence RASNLQS [SEQ ID NO: 14](IC14 L-CDR2); and L-CDR3 comprises the sequence QQSNEDPYT [SEQ ID NO: 27](IC14 L-CDR3); and b) an antibody VH domain, or antigen binding fragment thereof, comprising H-CDR1, H-CDR2 and H-CDR3, wherein: H-CDR1 comprises the sequence SDSAWN [SEQ ID NO: 16](IC14 H-CDR1); H-CDR2 comprises the sequence YISYSGSTSYNPSLKS [SEQ ID NO: 17](IC14 H-CDR2); and H-CDR3 comprises the sequence GLRFAY [SEQ ID NO: 18](IC14 H-CDR3); and
(iv) an antibody that comprises: a) an antibody VL domain, or antigen binding fragment thereof, comprising L-CDR1, L-CDR2 and L-CDR3, wherein: L-CDR1 comprises the sequence RASQDIKNYLN [SEQ ID NO: 19](18E12 L-CDR1); L-CDR2 comprises the sequence YTSRLHS [SEQ ID NO: 20](18E12 L-CDR2); and L-CDR3 comprises the sequence QRGDTLPWT [SEQ ID NO: 21](18E12 L-CDR3); and b) an antibody VH domain, or antigen binding fragment thereof, comprising H-CDR1, H-CDR2 and H-CDR3, wherein: H-CDR1 comprises the sequence NYDIS [SEQ ID NO: 22](18E12 H-CDR1); H-CDR2 comprises the sequence VIWTSGGTNYNSAFMS [SEQ ID NO: 23](18E12 H-CDR2); and H-CDR3 comprises the sequence GDGNFYLYNFDY [SEQ ID NO: 24](18E12 H-CDR3).

In particular examples, the CD14 antagonist antibody is selected from:
(i) an antibody comprising: a VL domain that comprises, consists or consists essentially of the sequence:

(3C10 VL)
[SEQ ID NO: 1]
QSPASLAVSLGQRATISCRASESVDSFGNSFMHWYQQKAGQPPKSSIYRA
ANLESGIPARFSGSGSRTDFTLTINPVEADDVATYFCQQSYEDPWTFGGG
TKLGNQ;

and a VH domain that comprises, consists or consists essentially of the sequence:

(3C10 VH)
[SEQ ID NO: 2]
LVKPGGSLKLSCVASGFTFSSYAMSWVRQTPEKRLEWVASISSGGTTYY
PDNVKGRFTISRDNARNILYLQMSSLRSEDTAMYYCARGYYDYHYWGQG
TTLTVSS;

(ii) an antibody comprising: a VL domain that comprises, consists or consists essentially of the sequence:

(28C5 VL)
[SEQ ID NO: 3]
QSPASLAVSLGQRATISCRASESVDSYVNSFLHWYQQKPGQPPKLLIYR
ASNLQSGIPARFSGSGSRTDFTLTINPVEADDVATYCCQQSNEDPTTFG
GGTKLEIK;

and a VH domain that comprises, consists or consists essentially of the sequence:

(28C5 VH)
[SEQ ID NO: 4]
LQQSGPGLVKPSQSLSLTCTVTGYSITSDSAWNWIRQFPGNRLEWMGYI
SYSGSTSYNPSLKSRISITRDTSKNQFFLQLNSVTTEDTATYYCVRGLR
FAYWGQGTLVTVSA;

(iii) an antibody comprising: a VL domain that comprises, consists or consists essentially of the sequence:

(IC14 VL)
[SEQ ID NO: 25]
QSPASLAVSLGQRATISCRASESVDSYVNSFLHWYQQKPGQPPKLLIYR
ASNLQSGIPARFSGSGSRTDFTLTINPVEADDVATYYCQQSNEDPYTFG
GGTKLEIK;

and a VH domain that comprises, consists or consists essentially of the sequence:

(IC14 VH)
[SEQ ID NO: 26]
LQQSGPGLVKPSQSLSLTCTVTGYSITSDSAWNWIRQFPGNRLEWMGYI
SYSGSTSYNPSLKSRISITRDTSKNQFFLQLNSVTTEDTATYYCVRGLR
FAYWGQGTLVTVSS;

and
(iv) an antibody comprising: a VL domain that comprises, consists or consists essentially of the sequence:

(18E12 VL)
[SEQ ID NO: 5]
QTPSSLSASLGDRVTISCRASQDIKNYLNWYQQPGGTVKVLIYYTSRLH
SGVPSRFSGSGSGTDYSLTISNLEQEDFATYFCQRGDTLPWTFGGGTKL
EIK;

and a VH domain that comprises, consists or consists essentially of the sequence:

(18E12 VH)
[SEQ ID NO: 6]
LESGPGLVAPSQSLSITCTVSGFSLTNYDISWIRQPPGKGLEWLGVIWT
SGGTNYNSAFMSRLSITKDNSESQVFLKMNGLQTDDTGIYYCVRGDGNF
YLYNFDYWGQGTTLTVSS.

In some embodiments, the CD14 antagonist antibody is humanized or chimeric.

In a particular example, the CD14 antagonist antibody comprises: a light chain comprising the amino acid sequence

[SEQ ID NO: 32]
DIVLTQSPASLAVSLGQRATISCRASESVDSYVNSFLHWYQQKPGQPPKLLIYRASNLQSGIPARFSGSG
SRTDFTLTINPVEADDVATYYCQQSNEDPYTFGGGTKLEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVT
KSFNRGEC;
and
a heavy chain comprising the amino acid sequence:
[SEQ ID NO: 33]
DVQLQQSGPGLVKPSQSLSLTCTVTGYSITSDSAWNWIRQFPGNRLEWMGYISYSGSTSYNPSLKSRIS
ITRDTSKNQFFLQLNSVTTEDTATYYCVRGLRFAYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVD
KRVESKYGPPCPSCPAPEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDKSRWQEGNVFS
CSVMHEALHNHYTQKSLSLSLGK.

In particular embodiments, the CD14 antagonist antibody is IC14.

The CD14 antagonist antibody may be administered in combination with (e.g. simultaneously or sequentially), or formulated with, an ancillary agent. The ancillary agent may be, for example, selected from among a fibrinolytic agent, beta blocker, high intensity statin, angiotensin converting enzyme (ACE) inhibitor and platelet inhibitor. In some examples, the fibrinolytic agent is selected from among streptokinase, anistreplase and a tissue plasminogen activator (e.g. tenecteplase, reteplase or alteplase). In further examples, the beta blocker is selected from among acebutolol, atenolol, bisoprolol, metoprolol, nadolol, nebivolol and propranolol. In still further examples, the platelet inhibitor is selected from among aspirin, a P2Y12 inhibitors (e.g. ticlopidine, clopidogrel, ticagrelor or prasugrel) and glycoprotein IIb/IIIa receptor antagonists.

In some embodiments, PCI is performed on the subject. In such examples, the CD14 antagonist antibody may be administered within, for example, 72 hours of PCI.

### BRIEF DESCRIPTION OF THE DRAWINGS

Embodiments of the disclosure are described herein, by way of non-limiting example only, with reference to the following drawings.

FIG. 1 is a graphical representation of echocardiographic assessment of systolic function 7 days post-surgery. (A) Area change. (B) Ejection Fraction. *p<0.05. Mean±SE.

FIG. 2 is a graphical representation levels of IL-1β, TNFα, IL-6 and IL-8 from iPSC-derived M0 (M0) that were stimulated with LPS and IFNγ. Cultures were treated with IC14 mAb or isotype control mAb at concentrations of 0.01 to 1 μg/ml. Levels of IL-1β, TNFα, IL-6 and IL-8 transcript were measured by qPCR (M1). Arbitrary units of each mRNA were normalized to β-actin and expression relative to M0 cells plotted. Cultures were treated with IC14 mAb or isotype control mAb at concentrations of 0.01 to 1 μg/ml. (A) IL-1β. (B) TNFα. (C) IL-6. (D) IL-8.

FIG. 3 is a graphical representation of echocardiographic assessments of systolic function 7 days post-surgery. Area change (A), and Ejection Fraction (B). * p<0.05, ** p<0.01, *** P<0.001. Mean±SEM. (Groups: A: Isotype, B: 3× Dose anti-CD14 Ab, C: Saline, D: 2× Dose anti-CD14 Ab).

FIG. 4 is a graphical representation of echocardiographic assessments of systolic function 7 days post-surgery, with combined control groups (A: Isotype+C: Saline) and anti-CD14 Ab groups (B: 3× Dose anti-CD14 Ab+D: 2× Dose anti-CD14 Ab).

FIG. 5 is a graphical representation of echocardiographic assessments of stroke volume (A) and cardiac output (B) 7 days post-surgery *p<0.05, **p<0.01, ***P<0.001. Mean±SEM. (Groups: A: Isotype, B: 3× Dose anti-CD14 Ab, C: Saline, D: 2× Dose anti-CD14 Ab).

FIG. 6 is a graphical representation of haemodynamic assessment of left ventricular and arterial pressures at 7 days post-surgery. (Groups: A: Isotype, B: 3× Dose anti-CD14 Ab, C: Saline, D: 2× Dose anti-CD14 Ab). Tx=treatment group (i.e. B+D). *p<0.05, **p<0.01, ***P<0.001. Mean±SEM.

FIG. 7 is a graphical representation of representative left ventricular (LV) pressure-volume loops 7 days post-surgery. Each loop indicates volume and pressure measurements throughout one complete cardiac cycle. (A) Representative LV pressure-volume loop from isotype control group. (B) Representative LV pressure-volume loop from 3× Dose anti-CD14 group. (C) Representative LV pressure-volume loop from saline control group. (D) Representative LV pressure-volume loop from 2× Dose anti-CD14 group.

FIG. 8 is a graphical representation of non-lesion area (A) and lesion size (B) measured from mid-ventricular brightfield sections 7 days post surgery *p<0.05. Mean±SEM. (Groups: A: Isotype, B: 3× Dose anti-CD14 Ab, C: Saline, D: 2× Dose anti-CD14 Ab).

FIG. 9 shows representative slides of the left ventricle of hearts stained with Picrosirius Red (Pic Red), where collagen is shown by dark grey and myocardium is shown by light grey. (A) Isotype control group. (B) 3× Dose anti-CD14 group. (C) Saline control group. (D) 2× Dose anti-CD14 group.

FIG. 10 is a graphical representation of CD68 positivity measured from mid-ventricular immunofluorescently-stained sections. *p<0.05. Mean±SEM. (A) CD68 positivity of each group. (B) CD68 positivity of groups A+C versus B+D. (Groups: A: Isotype, B: 3× Dose anti-CD14 Ab, C: Saline, D: 2× Dose anti-CD14 Ab).

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

The terms “active agent” and “therapeutic agent” are used interchangeably herein and refer to agents that prevent, reduce or amelioriate at least one symptom of a disease or disorder.

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same formulation. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

The term “antagonist antibody” is used in the broadest sense, and includes an antibody that inhibits or decreases the biological activity of an antigen to which the antibody binds (e.g., CD14). For example, an antagonist antibody may partially or completely block interaction between a receptor (e.g., CD14) and a ligand (e.g., a DAMP or PAMP), or may practically decrease the interaction due to tertiary structure change or down regulation of the receptor. Thus, a CD14 antagonist antibody encompasses antibodies that bind to CD14 and that block, inhibit, nullify, antagonize, suppress, decrease or reduce (including significantly), in any meaningful degree, a CD14 agonist activity, including activation of downstream pathways such as Toll-like receptor (TLR) signaling pathways (e.g., TLR4 signaling pathway) and the TIR-domain-containing adapter-inducing IFN-β(TRIF) pathway, or elicitation of a cellular response (e.g., production of pro-inflammatory mediators including pro-inflammatory cytokines) to CD14 binding by a CD14 ligand (e.g., a DAMP or PAMP). In some examples, the antibody is monovalent and binds only to CD14. In other examples, the antibody is bivalent and binds to CD14 and another antigen.

The term “antibody” herein is used in the broadest sense and specifically covers naturally occurring antibodies, monoclonal antibodies, polyclonal antibodies, multispecific antibodies (e.g., bispecific antibodies), antibody fragments, or any other antigen-binding molecule so long as they exhibit the desired immuno-interactivity. A naturally occurring “antibody” includes within its scope an immunoglobulin comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised specific CH domains (e.g., CH1, CH2 and CH3). Each light chain is comprised of a light chain variable region (abbreviated herein as VL) and a light chain constant region. The light chain constant region is comprised of one domain, CL. The VH and VL regions can be further subdivided into regions of hypervariability, termed complementary determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The constant regions of the antibodies may mediate the binding of an immunoglobulin to host tissues or factors, including various cells of the immune system (e.g., effector cells) and the first component (C1q) of the classical complement system. The antibodies can be of any isotype (e.g., IgG, IgE, IgM, IgD, IgA and IgY), class (e.g., IgG1, IgG2, IgG3, IgG4, IgA1 and IgA2), subclass or modified version thereof (e.g., IgG1 isotype, which carries L234A and L235A double mutations IgG1-LALA)). The antibodies can be of any species, chimeric, humanized or human. In other embodiments, the antibody is a homomeric heavy chain antibody (e.g., camelid antibodies) which lacks the first constant region domain (CH1) but retains an otherwise intact heavy chain and is able to bind antigens through an antigen-binding domain. The variable regions of the heavy and light chains in the antibody-modular recognition domain (MRD) fusions will contain a functional binding domain that interacts with an antigen of interest.

The “variable domain” (variable domain of a light chain (VL), variable domain of a heavy chain (VH)) as used herein denotes each of the pair of light and heavy chain domains which are involved directly in binding the antibody to the antigen. The variable light and heavy chain domains have the same general structure and each domain comprises four FRs whose sequences are widely conserved, connected by three CDRs or “hypervariable regions”. The FRs adopt a β-sheet conformation and the CDRs may form loops connecting the β-sheet structure. The CDRs in each chain are held in their three-dimensional structure by the FRs and form together with the CDRs from the other chain the antigen binding site.

The term “antigen-binding portion” when used herein refer to the amino acid residues of an antibody which are responsible for antigen-binding generally, which generally comprise amino acid residues from the CDRs. Thus, “CDR” or “complementarity determining region” (also referred to as “hypervariable region”) are used interchangeably herein to refer to the amino acid sequences of the light and heavy chains of an antibody which form the three-dimensional loop structure that contributes to the formation of an antigen binding site. There are three CDRs in each of the variable regions of the heavy chain and the light chain, which are designated “CDR1”, “CDR2”, and “CDR3”, for each of the variable regions. The term “CDR set” as used herein refers to a group of three CDRs that occur in a single variable region that binds the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, 1987. J. Mol. Biol. 196: 901-917; Chothia et al., 1989. Nature 342: 877-883) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan (1995. FASEB J. 9: 133-139) and MacCallum (1996. J. Mol. Biol. 262(5): 732-745). Still other CDR boundary definitions may not strictly follow one of these systems, but will nonetheless overlap with the Kabat CDRs, although they may be shortened or lengthened in light of prediction or experimental findings that particular residues or groups of residues or even entire CDRs do not significantly impact antigen binding.

As used herein, the term “framework region” or “FR” refers to the remaining sequences of a variable region minus the CDRs. Therefore, the light and heavy chain variable domains of an antibody comprise from N- to C-terminus the domains FR1, CDR1, FR2, CDR2, FR3, CDR3, and FR4. CDRs and FRs are typically determined according to the standard definition of Kabat, E. A., et al., Sequences of Proteins of Immunological Interest, 5th ed., Public Health Service, National Institutes of Health, Bethesda, Md. (1991) and/or those residues from a “hypervariable loop”.

As used herein, the terms “light chain variable region” (“VL”) and “heavy chain variable region” (VH) refer to the regions or domains at the N-terminal portion of the light and heavy chains respectively which have a varied primary amino acid sequence for each antibody. The variable region of an antibody typically consists of the amino terminal domain of the light and heavy chains as they fold together to form a three-dimensional binding site for an antigen. Several subtypes of VH and VL, based on structural similarities, have been defined, for example as set forth in the Kabat database.

The term “chimeric antibody” refers to antibodies that comprise heavy and light chain variable region sequences from one species and constant region sequences from another species, such as antibodies having murine heavy and light chain variable regions linked to human constant regions.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Thus, the FRs and CDRs of a humanized antibody need not correspond precisely to the parental (i.e., donor) sequences, e.g., a donor antibody CDR or the consensus framework may be mutagenized by substitution, insertion, and/or deletion of at least one amino acid residue so that the CDR or FR at that site does not correspond to either the donor antibody or the consensus framework. Typically, such mutations, however, will not be extensive and will generally avoid “key residues” involved in binding to an antigen. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, for example, Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will generally comprise at least a portion of an immunoglobulin constant region (Fc), typically that of a human immunoglobulin. For further details, see Jones et al. (1986. Nature 321:522-525), Riechmann et al. (1988. Nature 332:323-329) and Presta (1992. Curr. Op. Struct. Biol. 2:593-596). A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well known in the art. As used herein, the term “key residue” refers to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

As used herein, “Vernier” zone refers to a subset of framework residues that may adjust CDR structure and fine-tune the fit to antigen as described by Foote and Winter (1992. J. Mol. Biol. 224: 487-499). Vernier zone residues form a layer underlying the CDRs and may impact on the structure of CDRs and the affinity of the antibody.

As used herein, the term “canonical” residue refers to a residue in a CDR or framework that defines a particular canonical CDR structure as defined by Chothia et al. (1987. J. Mol. Biol. 196: 901-917; 1992. J. Mol. Biol. 227: 799-817), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs to an “acceptor antibody”. In some embodiments, the donor antibody is an antibody from a species different from the antibody from which the FRs are obtained or derived. In the context of a humanized antibody, the term “donor antibody” refers to a non-human antibody providing one or more CDRs.

As used herein, the terms “acceptor” and “acceptor antibody” refer to an antibody providing at least 80%, at least 85%, at least 90%, at least 95%, at least 98%, or 100% of the amino acid sequences of one or more of the FRs. In some embodiments, the term “acceptor” refers to the antibody amino acid sequence providing the constant region(s). In other embodiments, the term “acceptor” refers to the antibody amino acid sequence providing one or more of the FRs and the constant region(s). In specific embodiments, the term “acceptor” refers to a human antibody amino acid sequence that provides at least 80%, preferably, at least 85%, at least 90%, at least 95%, at least 98%, or 100% of the amino acid sequences of one or more of the FRs. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, for example, derived or obtained from a germline antibody gene, a mature antibody gene, a functional antibody (e.g., antibodies well-known in the art, antibodies in development, or antibodies commercially available).

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The terms “heavy chain variable region CDR1” and “H-CDR1” are used interchangeably, as are the terms “heavy chain variable region CDR2” and “H-CDR2”, the terms “heavy chain variable region CDR3” and “H-CDR3”, the terms “light chain variable region CDR1” and “L-CDR1”; the terms “light chain variable region CDR2” and “L-CDR2” and the terms “light chain variable region CDR3” and “L-CDR3” antibody fragment. Throughout the specification, complementarity determining regions (“CDR”) are defined according to the Kabat definition unless specified otherwise. The Kabat definition is a standard for numbering the residues in an antibody and it is typically used to identify CDR regions (Kabat et al., (1991), 5th edition, NIH publication No. 91-3242).

Antigen binding can be performed by “fragments” or “antigen-binding fragments” of an intact antibody. Herein, both terms are used interchangeably. Examples of binding fragments encompassed within the term “antibody fragment” of an antibody include a Fab fragment, a monovalent fragment consisting of the VL, VH, CL and CH1 domains; a F(ab′)2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989. Nature 341:544-546), which consists of a VH domain; and an isolated complementary determining region (CDR). In a particular embodiment, the antibody of the present disclosure is an antigen-binding fragment that lacks all or a portion of the Fc region.

A “single chain variable Fragment (scFv)” is a single protein chain in which the VL and VH regions pair to form monovalent molecules (known as single chain Fv (scFv); see, e.g., Bird et al., 1988. Science 242:423-426; and Huston et al., 1988. Proc. Natl. Acad. Sci. 85:5879-5883). Although the two domains VL and VH are coded for by separate genes, they can be joined, using recombinant methods, by an artificial peptide linker that enables them to be made as a single protein chain. Such single chain antibodies include one or more antigen binding moieties. These antibody fragments are obtained using conventional techniques known to those of skill in the art, and the fragments are screened for utility in the same manner as are intact antibodies.

The term “monoclonal antibody” and abbreviations “MAb” and “mAb”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method. Monoclonal antibodies may be produced, for example, by a single clone of antibody-producing cells, including hybridomas. The term “hybridoma” generally refers to the product of a cell-fusion between a cultured neoplastic lymphocyte and a primed B- or T-lymphocyte which expresses the specific immune potential of the parent cell.

An antibody “that binds” an antigen of interest (e.g., CD14) is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody, oligopeptide or other organic molecule to its particular target protein as determined, for example, by fluorescence activated cell sorting (FACS) analysis, enzyme-linked immunosorbent assay (ELISA), immunoprecipitation or radioimmunoprecipitation (RIA). Thus, an antibody that antagonizes CD14 suitably inhibits or decreases production of pro-inflammatory mediators, including pro-inflammatory cytokines/chemokines. With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The specific region of the antigen to which the antibody binds is typically referred to as an “epitope”. The term “epitope” broadly includes the site on an antigen which is specifically recognized by an antibody or T-cell receptor or otherwise interacts with a molecule. Generally epitopes are of active surface groupings of molecules such as amino acids or carbohydrate or sugar side chains and generally may have specific three-dimensional structural characteristics, as well as specific charge characteristics. As will be appreciated by one of skill in the art, practically anything to which an antibody can specifically bind could be an epitope.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “effective amount”, in the context of treating a disease or condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the age, health and physical condition of the individual to be treated and whether symptoms of disease are apparent, the taxonomic group of individual to be treated, the formulation of the composition, the assessment of the medical situation, and other relevant factors. Optimal dosing schedules can be calculated from measurements of drug accumulation in the body of the subject. Optimum dosages may vary depending on the relative potency in an individual subject, and can generally be estimated based on EC50 values found to be effective in in vitro and in vivo animal models. Persons of ordinary skill can easily determine optimum dosages, dosing methodologies and repetition rates. It is expected that the amount will fall in a relatively broad range that can be determined through routine trials.

The terms “increase” or “increasing” and the like, in relation to systolic function (or ventricular function) refers to at least a small but measurable increase in the systolic function of a subject with a MI following administration of an anti-CD14 antagonist antibody, compared to a subject with a MI that has not been administered the antibody. Typically, the increase is a statistically significant increase. In some embodiments, systolic function is increased by at least 20%, 30%, 40%, 50%, 60%, 70% 80%, 90%, 100%, 150%, 200% or more. Conversely, “decrease”, “decreasing”, “reduce” or “reducing” and the like, in relation to systolic dysfunction (or ventricular dysfunction) refers to at least a small but measurable decrease or reduction in the systolic dysfunction of a subject with a MI following administration of an anti-CD14 antagonist antibody, compared to a subject with a MI that has not been administered the antibody. Typically, the decrease is a statistically significant decrease. In some embodiments, systolic function is decreased by at least 20%, 30%, 40%, 50%, 60%, 70% 80% or more. Systolic function (or dysfunction) can be assessed using any method known in the art. In one example, systolic function is assessed by echocardiography, where one or more 2-dimensional or 3-dimensional parameters (e.g. end-diastolic area, end-systolic area, area change, longitudinal fractional shortening, end-diastolic volume, end-systolic volume, stroke volume, cardiac output, and/or ejection fraction) are used as an indication of systolic function, such as demonstrated in the examples below.

By “isolated” is meant material that is substantially or essentially free from components that normally accompany it in its native state.

The term “ligand”, as used herein, refers to any molecule which is capable of binding a receptor.

The term “myocardial infarction” or MI refers to tissue death (i.e. infarction) of the heart muscle, or the myocardium, as a result of ischaemia. MI can be diagnosed by those skilled in the art based on accepted criteria, such as set out in the Fourth Universal Definition of Myocardial Infarction (Thygesen et al. 2018, Circulation, 138: e618-e651). For example, MI may be diagnosed in a clinical setting when the presence of acute myocardial injury is detected by abnormal cardiac biomarkers (e.g. cardiac troponin I (cTnI) and T (cTnT), whereby myocardial injury is defined as being present when blood levels of cTn are increased above the 99th percentile upper reference limit (URL)) with evidence of acute myocardial ischemia (e.g. as indicated by ECG or the presence of ischemic symptoms such as chest, upper extremity, mandibular, or epigastric discomfort during exertion or at rest, or an ischemic equivalent such as dyspnea or fatigue).

MI can be classified into types based on etiology and circumstances: Type 1: Spontaneous MI caused by ischemia due to a primary coronary event (e.g., plaque rupture, erosion, or fissuring; coronary dissection; Type 2: Ischemia due to increased oxygen demand (e.g., hypertension), or decreased supply (e.g., coronary artery spasm or embolism, arrhythmia, hypotension); Type 3: Related to sudden unexpected cardiac death; Type 4a: Associated with percutaneous coronary intervention (signs and symptoms of myocardial infarction with cTn values>5×99th percentile URL) Type 4b: Associated with documented stent thrombosis; and Type 5: Associated with coronary artery bypass grafting (signs and symptoms of myocardial infarction with cTn values>10×99th percentile URL).

MI can also be classified as ST-segment elevation myocardial infarction (STEMI) or non-ST-segment-elevation myocardial infarction (non-STEMI) by the presence or absence, respectively, of ST-segment elevation or Q waves on the ECG.

The term “post-MI” with reference to a time period means the time period after the onset of the first symptom(s) of MI (e.g. pressure or tightness in the chest; pain in the chest, back, jaw, and other areas of the upper body; shortness of breath). Thus, for example, reference to “12 hours post-MI” means 12 hours after the onset of MI symptoms.

The term “post-MI diagnosis” with reference to a time period means the time period after the diagnosis of MI, such as by a medical practitioner in a hospital or other medical facility. Thus, for example, reference to “12 hours post-MI diagnosis” means 12 hours after the diagnosis of MI.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, transfection agents and the like.

Similarly, a “pharmacologically acceptable” salt, ester, amide, prodrug or derivative of a compound as provided herein is a salt, ester, amide, prodrug or derivative that this not biologically or otherwise undesirable.

The terms “polynucleotide,” “genetic material,” “genetic forms,” “nucleic acids” and “nucleotide sequence” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art.

The term “pro-inflammatory mediator” means an immunoregulatory agent that favors inflammation. Such agents include, cytokines such as chemokines, interleukins (IL), lymphokines, and tumor necrosis factor (TNF) as well as growth factors. In specific embodiments, the pro-inflammatory mediator is a “pro-inflammatory cytokine”. Typically, pro-inflammatory cytokines include IL-1α, IL-1β, IL-6, and TNF-α, which are largely responsible for early responses. Other pro-inflammatory mediators include LIF, IFN-γ, IFN-β, IFN-α, OSM, CNTF, TGF-β, GM-CSF, TWEAK, IL-11, IL-12, IL-15, IL-17, IL-18, IL-19, IL-20, IL-8, IL-16, IL-22, IL-23, IL-31 and IL-32 (Tato et al., 2008. Cell 132:900; Cell 132:500, Cell 132:324). Pro-inflammatory mediators may act as endogenous pyrogens (IL-1, IL-6, IL-17, TNF-α), up-regulate the synthesis of secondary mediators and pro-inflammatory cytokines by both macrophages and mesenchymal cells (including fibroblasts, epithelial and endothelial cells), stimulate the production of acute phase proteins, or attract inflammatory cells. In specific embodiments, the term “pro-inflammatory cytokine” relates to TNF-α, IL-1α, IL-6, IFNβ, IL-1β, IL-8, IL-17 and IL-18.

Reference herein to a “single dose” of a CD14 antagonist antibody means that the subject is administered only one dose, e.g. in one bolus injection or one discrete infusion, of the antibody following MI. In the event that the subject suffers a further MI, the subject may be administered a single dose of the antibody for that further MI. Thus, reference to a single dose means that the subject receives only one dose of the antibody for each instance of MI.

As used herein, the term “systemic administration” or “administered systemically” or “systemically administered” means introducing an agent into a subject outside of the central nervous system. Systemic administration encompasses any route of administration other than direct administration to the spine or brain. As such, it is clear that intrathecal and epidural administration as well as cranial injection or implantation, are not within the scope of the terms “systemic administration”, “administered systemically” or “systemically administered”. An agent (e.g. an antibody) or pharmaceutical composition as described herein can be systemically administered in any acceptable form such as in a tablet, liquid, capsule, powder, or the like; by intravenous, intraperitoneal, intramuscular, subcutaneous or parenteral injection; by transdermal diffusion or electrophoresis; and by minipump or other implanted extended release device or formulation. According to some embodiments, systemic administration is carried out by a route selected from the group consisting of intraperitoneal, intravenous, subcutaneous and intranasal administration, and combinations thereof.

The terms “subject”, “patient” and “individual” used interchangeably herein, refer to any subject, particularly a vertebrate subject, and even more particularly a mammalian subject, (e.g. human) with a MI.

As used herein, the terms “treatment”, “treating”, and the like, refer to obtaining a desired pharmacologic and/or physiologic effect in a subject in need of treatment, that is, a subject who has a MI. By “treatment” is meant ameliorating or preventing one or more symptoms or effects (e.g. consequences) of MI. In particular examples, treatment includes ameliorating or preventing damage to the heart muscle (e.g. myocardium; e.g. limiting infarct size, limiting or preventing fibrosis), and/or ameliorating or preventing a reduction in heart function (e.g. systolic function, contractile properties, haemodynamic function, etc.). Reference to “treatment”, “treat” or “treating” does not necessarily mean to reverse or prevent any or all symptoms or effects of MI. For example, the subject may ultimately suffer one or more symptoms or effects, but the number and/or severity of the symptoms or effects is reduced and/or the function of the heart is improved or the quality of life is improved compared to in the absence of treatment.

Each embodiment described herein is to be applied mutatis mutandis to each and every embodiment unless specifically stated otherwise.

### 2. CD14 Antagonist Antibodies

The present disclosure provides methods, uses and compositions that include a CD14 antagonist antibody for treating MI in a subject. The present disclosure also provides methods, uses and compositions that include a CD14 antagonist antibody for treating MI.

The present disclosure contemplates any CD14 antagonist antibody that binds to CD14, such as human CD14 (e.g. human mCD14 or sCD14) and blocks the binding of a DAMP or PAMP to CD14 and/or that binds to CD14 and inhibits or decreases a CD14 agonist-mediated response resulting in the production of pro-inflammatory mediators, including the production of pro-inflammatory cytokines. Such CD14 antagonist antibodies are well known in the art and any can be utilized in the methods and uses of the present disclosure. In some embodiments, a CD14 antagonist antibody of the present invention inhibits binding of a CD14 agonist, suitably a DAMP or PAMP, to CD14 thus inhibiting or decreasing the production of pro-inflammatory cytokines. In illustrative examples of this type, the CD14 antagonist antibody is selected from the 3C10 antibody that binds an epitope comprised in at least a portion of the region from amino acid 7 to amino acid 14 of human CD14 (van Voohris et al., 1983. J. Exp. Med. 158: 126-145; Juan et al., 1995. J. Biol. Chem. 270(29): 17237-17242), the MEM-18 antibody that binds an epitope comprised in at least a portion of the region from amino acid 57 to amino acid 64 of CD14 (Bazil et al., 1986. Eur. J. Immunol. 16(12):1583-1589; Juan et al., 1995. J. Biol. Chem. 270(10): 5219-5224), the 4C1 antibody (Adachi et al., 1999. J. Endotoxin Res. 5: 139-146; Tasaka et al., 2003. Am. J. Respir. Cell. Mol. Biol.; 2003. 29(2):252-258), as well as the 28C5 and 23G4 antibodies that inhibit binding of LPS and suppress production of pro-inflammatory cytokines, and the 18E12 antibody that partly inhibits binding of LPS and suppresses production of pro-inflammatory cytokines (U.S. Pat. Nos. 5,820,858, 6,444,206 and 7,326,569 to Leturcq et al.). In some embodiments, a CD14 antagonist antibody of the present disclosure inhibits binding of CD14 to a TLR such as TLR4, thereby blocking CD14-agonist mediated response, illustrative examples of which include the F1024 antibody disclosed in International Publication WO2002/42333. Other CD14 antagonist antibodies include the single-chain antibody scFv2F9 and the related human-mouse chimeric antibody Hm2F9 (Tang et al. 2007, Immunopharmacol Immunotoxicol 29,375-386; and Shen et al., 2014, DNA Cell Biol. 33(9): 599-604). Further examples of CD14 antagonist antibodies include the anti-human CD14 18D11 IgG1 mAb, 18D11 IgG1 F(ab)′2 fragment and the chimeric r18D11 antibody (IgG2/4) (see e.g. Lau et al., 2013, J Immunol 191:4769-4777). Each of the above references relating to CD14 antagonist antibodies is incorporated herein by reference in its entirety. The CD14 antagonist antibody may be a full-length immunoglobulin antibody or an antigen-binding fragment of an intact antibody, representative examples of which include a Fab fragment, a F(ab′)2 fragment, an Fd fragment consisting of the VH and CH1 domains, an Fv fragment consisting of the VL and VH domains of a single arm of an antibody, a single domain antibody (dAb) fragment (Ward et al., 1989. Nature 341:544-546), which consists of a VH domain; and an isolated CDR. Suitably, the CD14 antagonist antibody is a chimeric, humanized or human antibody.

In some embodiments, the CD14 antagonist antibody comprises a VH and VL of an antibody disclosed in U.S. Pat. No. 5,820,858:
(1) an antibody comprising:
a VL domain comprising, consisting or consisting essentially of the sequence:

(3C10 VL)
[SEQ ID NO: 1]
QSPASLAVSLGQRATISCRASESVDSFGNSFMHWYQQKAGQPPKSSIYR
AANLESGIPARFSGSGSRTDFTLTINPVEADDVATYFCQQSYEDPWTFG
GGTKLGNQ;

and
a VH domain comprising, consisting or consisting essentially of the sequence:

(3C10 VH)
[SEQ ID NO: 2]
LVKPGGSLKLSCVASGFTFSSYAMSWVRQTPEKRLEWVASISSGGTTYY
PDNVKGRFTISRDNARNILYLQMSSLRSEDTAMYYCARGYYDYHYWGQG
TTLTVSS;

(2) an antibody comprising:
a VL domain comprising, consisting or consisting essentially of the sequence: NA,

(28C5 VL)
[SEQ ID NO: 3]
QSPASLAVSLGQRATISCRASESVDSYVNSFLHWYQQKPGQPPKLLIYR
ASNLQSGIPARFSGSGSRTDFTLTINPVEADDVATYCCQQSNEDPTTFG
GGTKLEIK;

and
a VH domain comprising, consisting or consisting essentially of the sequence:

(28C5 VH)
[SEQ ID NO: 4]
LQQSGPGLVKPSQSLSLTCTVTGYSITSDSAWNWIRQFPGNRLEWMGYI
SYSGSTSYNPSLKSRISITRDTSKNQFFLQLNSVTTEDTATYYCVRGLR
FAYWGQGTLVTVSA;

and
(3) an antibody comprising:
a VL domain comprising, consisting or consisting essentially of the sequence:

(18E12 VL)
[SEQ ID NO: 5]
QTPSSLSASLGDRVTISCRASQDIKNYLNWYQQPGGTVKVLIYYTSRLH
SGVPSRFSGSGSGTDYSLTISNLEQEDFATYFCQRGDTLPWTFGGGTKL
EIK;

and
a VH domain comprising, consisting or consisting essentially of the sequence:

(18E12 VH)
[SEQ ID NO: 6]
LESGPGLVAPSQSLSITCTVSGFSLTNYDISWIRQPPGKGLEWLGVIWT
SGGTNYNSAFMSRLSITKDNSESQVFLKMNGLQTDDTGIYYCVRGDGNF
YLYNFDYWGQGTTLTVSS;

Also contemplated are antibodies that comprise the VL and VH CDR sequences of the above antibodies and related antibodies, representative embodiments of which include:
(1) an antibody that comprises: a) an antibody VL domain, or antigen binding fragment thereof, comprising L-CDR1, L-CDR2 and L-CDR3, wherein: L-CDR1 comprises the sequence RASESVDSFGNSFMH [SEQ ID NO: 7](3C10 L-CDR1); L-CDR2 comprises the sequence RAANLES [SEQ ID NO: 8](3C10 L-CDR2); and L-CDR3 comprises the sequence QQSYEDPWT [SEQ ID NO: 9](3C10 L-CDR3); and b) an antibody VH domain, or antigen binding fragment thereof, comprising H-CDR1, H-CDR2 and H-CDR3, wherein: H-CDR1 comprises the sequence SYAMS [SEQ ID NO: 10](3C10 H-CDR1); H-CDR2 comprises the sequence SISSGGTTYYPDNVKG [SEQ ID NO: 11](3C10 H-CDR2); and H-CDR3 comprises the sequence GYYDYHY [SEQ ID NO: 12](3C10 H-CDR3);
(2) an antibody that comprises: a) an antibody VL domain, or antigen binding fragment thereof, comprising L-CDR1, L-CDR2 and L-CDR3, wherein: L-CDR1 comprises the sequence RASESVDSYVNSFLH [SEQ ID NO: 13](28C5 L-CDR1); L-CDR2 comprises the sequence RASNLQS [SEQ ID NO: 14](28C5 L-CDR2); and L-CDR3 comprises the sequence QQSNEDPTT [SEQ ID NO: 15](28C5 L-CDR3); and b) an antibody VH domain, or antigen binding fragment thereof, comprising H-CDR1, H-CDR2 and H-CDR3, wherein: H-CDR1 comprises the sequence SDSAWN [SEQ ID NO: 16](28C5 H-CDR1); H-CDR2 comprises the sequence YISYSGSTSYNPSLKS [SEQ ID NO: 17](28C5 H-CDR2); and H-CDR3 comprises the sequence GLRFAY [SEQ ID NO: 18](28C5 H-CDR3);
(3) an antibody that comprises: a) an antibody VL domain, or antigen binding fragment thereof, comprising L-CDR1, L-CDR2 and L-CDR3, wherein: L-CDR1 comprises the sequence RASESVDSYVNSFLH [SEQ ID NO: 13](IC14 L-CDR1); L-CDR2 comprises the sequence RASNLQS [SEQ ID NO: 14](IC14 L-CDR2); and L-CDR3 comprises the sequence QQSNEDPYT [SEQ ID NO: 27](IC14 L-CDR3); and b) an antibody VH domain, or antigen binding fragment thereof, comprising H-CDR1, H-CDR2 and H-CDR3, wherein: H-CDR1 comprises the sequence SDSAWN [SEQ ID NO: 16](IC14 H-CDR1); H-CDR2 comprises the sequence YISYSGSTSYNPSLKS [SEQ ID NO: 17](IC14 H-CDR2); and H-CDR3 comprises the sequence GLRFAY [SEQ ID NO: 18](IC14 H-CDR3); and
(4) an antibody that comprises: a) an antibody VL domain, or antigen binding fragment thereof, comprising L-CDR1, L-CDR2 and L-CDR3, wherein: L-CDR1 comprises the sequence RASQDIKNYLN [SEQ ID NO: 19](18E12 L-CDR1); L-CDR2 comprises the sequence YTSRLHS [SEQ ID NO: 20](18E12 L-CDR2); and L-CDR3 comprises the sequence QRGDTLPWT [SEQ ID NO: 21](18E12 L-CDR3); and b) an antibody VH domain, or antigen binding fragment thereof, comprising H-CDR1, H-CDR2 and H-CDR3, wherein: H-CDR1 comprises the sequence NYDIS [SEQ ID NO: 22](18E12 H-CDR1); H-CDR2 comprises the sequence VIWTSGGTNYNSAFMS [SEQ ID NO: 23](18E12 H-CDR2); and H-CDR3 comprises the sequence GDGNFYLYNFDY [SEQ ID NO: 24](18E12 H-CDR3).

In some embodiments, the CD14 antagonist antibody is humanized. In illustrative examples of this type, the humanized CD14 antagonist antibodies suitably comprise a donor CDR set corresponding to a CD14 antagonist antibody (e.g., one of the CD14 antagonist antibodies described above), and a human acceptor framework. The human acceptor framework may comprise at least one amino acid substitution relative to a human germline acceptor framework at a key residue selected from the group consisting of: a residue adjacent to a CDR; a glycosylation site residue; a rare residue; a canonical residue; a contact residue between heavy chain variable region and light chain variable region; a residue within a Vernier zone; and a residue in a region that overlaps between a Chothia-defined VH CDR1 and a Kabat-defined first heavy chain framework. Techniques for producing humanized mAbs are well known in the art (see, for example, Jones et al., 1986. Nature 321: 522-525; Riechmann et al. 1988. Nature 332:323-329; Verhoeyen et al., 1988. Science 239: 1534-1536; Carter et al., 1992. Proc. Natl. Acad. Sci. USA 89: 4285-4289; Sandhu, J S., 1992. Crit. Rev. Biotech. 12: 437-462, and Singer et al., 1993. J. Immunol. 150: 2844-2857). A chimeric or murine monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. As simply transferring mouse CDRs into human FRs often results in a reduction or even loss of antibody affinity, additional modification might be required in order to restore the original affinity of the murine antibody. This can be accomplished by the replacement of one or more human residues in the FR regions with their murine counterparts to obtain an antibody that possesses good binding affinity to its epitope. See, for example, Tempest et al. (1991. Biotechnology 9:266-271) and Verhoeyen et al. (1988 supra). Generally, those human FR amino acid residues that differ from their murine counterparts and are located close to or touching one or more CDR amino acid residues would be candidates for substitution.

In one embodiment, the CD14 antagonist antibody is the IC14 antibody (Axtelle et al., 2001. J. Endotoxin Res. 7: 310-314; and U.S. Pat. Appl. No. 2006/0121574, which are incorporated herein by reference in their entirety) or an antigen-binding fragment thereof. The IC14 antibody is a chimeric (murine/human) monoclonal antibody that specifically binds to human CD14. IC14 was derived from the murine 28C5 noted above and comprises an IgG4 heavy chain (see, U.S. Pat. Nos. 5,820,858, 6,444,206 and 7,326,569 to Leturcq et al., and Leturcq et al., 1996. J. Clin. Invest. 98: 1533-1538). Thus, in one example, the CD14 antagonist antibody comprises the IC14 heavy chain and light chain CDRs, as described above. In another example, the CD14 antagonist antibody comprises the VL domain and a VH domain, wherein:
the VL domain comprises the amino acid sequence:

[SEQ ID NO: 25]
QSPASLAVSLGQRATISCRASESVDSYVNSFLHWYQQKPGQPPKLLIYR
ASNLQSGIPARFSGSGSRTDFTLTINPVEADDVATYYCQQSNEDPYTFG
GGTKLEIK;

and
the VH domain comprises the amino acid sequence:

[SEQ ID NO: 26]
LQQSGPGLVKPSQSLSLTCTVTGYSITSDSAWNWIRQFPGNRLEWMGYI
SYSGSTSYNPSLKSRISITRDTSKNQFFLQLNSVTTEDTATYYCVRGLR
FAYWGQGTLVTVSS;

or
the VL domain comprises the amino acid sequence:

[SEQ ID NO: 30]
DIVLTQSPASLAVSLGQRATISCRASESVDSYVNSFLHWYQQKPGQPPK
LLIYRASNLQSGIPARFSGSGSRTDFTLTINPVEADDVATYYCQQSNED
PYTFGGGTKLEIK;

and
the VH domain comprises the amino acid sequence:

[SEQ ID NO: 31]
DVQLQQSGPGLVKPSQSLSLTCTVTGYSITSDSAWNWIRQFPGNR
LEWMGYISYSGSTSYNPSLKSRISITRDTSKNQFFLQLNSVTTED
TATYYCVRGLRFAYWGQGTLVTVSS.

In another example, the CD14 antagonist antibody comprises light chain and heavy chain of IC14, wherein:
the light chain comprises the amino acid sequence:

[SEQ ID NO: 28]
QSPASLAVSLGQRATISCRASESVDSYVNSFLHWYQQKPGQPPKL
LIYRASNLQSGIPARFSGSGSRTDFTLTINPVEADDVATYYCQQS
NEDPYTFGGGTKLEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC;

and
the heavy chain comprises the amino acid sequence:

[SEQ ID NO: 29]
LQQSGPGLVKPSQSLSLTCTVTGYSITSDSAWNWIRQFPGNRLEW
MGYISYSGSTSYNPSLKSRISITRDTSKNQFFLQLNSVTTEDTAT
YYCVRGLRFAYWGQGTLVTVSSASTKGPSVFPLAPCSRSTSESTA
ALGCLVKDYFPEPVTVSWNSGALTSGVHTFPAVLQSSGLYSLSSV
VTVPSSSLGTKTYTCNVDHKPSNTKVDKRVESKYGPPCPSCPAPE
FLGGPSVFLFPPKPKDTLMISRTPEVTCVVVDVSQEDPEVQFNWY
VDGVEVHNAKTKPREEQFNSTYRVVSVLTVLHQDWLNGKEYKCKV
SNKGLPSSIEKTISKAKGQPREPQVYTLPPSQEEMTKNQVSLTCL
VKGFYPSDIAVEWESNGQPENNYKTTPPVLDSDGSFFLYSRLTVD
KSRWQEGNVFSCSVMHEALHNHYTQKSLSLSLGK;

or
the light chain comprises the amino acid sequence:

[SEQ ID NO: 32]
DIVLTQSPASLAVSLGQRATISCRASESVDSYVNSFLHWYQQKPG
QPPKLLIYRASNLQSGIPARFSGSGSRTDFTLTINPVEADDVATY
YCQQSNEDPYTFGGGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC;

and
the heavy chain comprises the amino acid sequence

[SEQ ID NO: 33]
DVQLQQSGPGLVKPSQSLSLTCTVTGYSITSDSAWNWIRQFPGNR
LEWMGYISYSGSTSYNPSLKSRISITRDTSKNQFFLQLNSVTTED
TATYYCVRGLRFAYWGQGTLVTVSSASTKGPSVFPLAPCSRSTSE
STAALGCLVKDYFPEPVTVSWNSGALTSGVHTFPAVLQSSGLYSL
SSVVTVPSSSLGTKTYTCNVDHKPSNTKVDKRVESKYGPPCPSCP
APEFLGGPSVFLFPPKPKDTLMISRTPEVTCVVVDVSQEDPEVQF
NWYVDGVEVHNAKTKPREEQFNSTYRVVSVLTVLHQDWLNGKEYK
CKVSNKGLPSSIEKTISKAKGQPREPQVYTLPPSQEEMTKNQVSL
TCLVKGFYPSDIAVEWESNGQPENNYKTTPPVLDSDGSFFLYSRL
TVDKSRWQEGNVFSCSVMHEALHNHYTQKSLSLSLGK.

Additional antagonist antibodies of CD14 suitable for use in the methods herein can be identified by methods well known to those skilled in the art. These methods generally comprise determining whether an antibody is capable of directly antagonizing CD14. For example, the methods may involve determining whether an antibody is capable of inhibiting or decreasing the amount or agonist activity of CD14, wherein the ability to inhibit or decrease the amount or agonist activity of CD14 indicates that the antibody may be suitable for use in treating MI. In some embodiments, the antibody is contacted with CD14, or a cell that expresses CD14 on its surface, or a nucleic acid sequence from which CD14 is expressed, suitably in the presence of a CD14 agonist such as a DAMP or PAMP, wherein a decrease in the amount or agonist activity of CD14 in the presence of the agonist, when compared to a control, indicates that the antibody binds to CD14 and directly antagonizes CD14. A decrease or inhibition of CD14 agonist activity, includes for example inhibiting, or decreasing activation of, downstream pathways such as TLR signaling pathways (e.g., TLR4 signaling pathway) and the TRIF pathway, or elicitation of a cellular response (e.g., production of pro-inflammatory mediators including pro-inflammatory cytokines).

These methods may be carried out in vivo, ex vivo or in vitro. In particular, the step of contacting an antibody with CD14 or with a cell that expresses CD14 on its surface (e.g., immune cells) may be carried out in vivo, ex vivo or in vitro. The methods may be carried out in a cell-based or a cell-free system. For example, the method may comprise a step of contacting a cell expressing CD14 on its surface with an antibody and determining whether the contacting of the cell with the antibody leads to a decrease in the amount or agonist activity of CD14. In such a cell-based assay, the CD14 and/or the antibody may be endogenous to the host cell, may be introduced into a host cell or tissue, may be introduced into the host cell or tissue by causing or allowing the expression of an expression construct or vector or may be introduced into the host cell by stimulating or activating expression from an endogenous gene in the cell. In such a cell-based method, the amount of activity of CD14 may be assessed in the presence or absence of an antibody in order to determine whether the agent is altering the amount of CD14 in the cell, such as through regulation of CD14 expression in the cell or through destabilization of CD14 protein within the cell, or altering the CD14 agonist activity of the cell. The presence of a lower CD14 agonist activity or a decreased amount of CD14 on the cell surface in the presence of the antibody indicates that the antibody may be a suitable antagonist of CD14 for use in accordance with the present disclosure.

In some examples, it is further determined whether the antibody lacks substantial or detectable binding to another cellular component, suitably a binding partner of CD14, such as a CD14 binding partner that is either secreted (e.g., MD2) or located on the cell membrane (e.g., TLR4), to thereby determine that the antibody is a specific antagonist of CD14. In a non-limiting example of this type, the antibody is contacted in the presence of a CD14 agonist such as a DAMP or PAMP (1) with a wild-type cell that expresses CD14 on its surface (e.g., an immune cell such a macrophage), and (2) with a CD14 negative cell (e.g., an immune cell that is the same as in (1) but has a loss of function in the CD14 gene). If the antibody inhibits a CD14 agonist activity of the wild-type cell but not of the CD14 negative cell, this indicates that the antibody is a CD14 specific antagonist. Cells of this type may be constructed using routine procedures or animals.

In other examples, potential CD14 antagonist antibodies are assessed in vivo, such as, for example, in an animal model. In such an in vivo model, the effects of the antibody may be assessed in the circulation (e.g., blood) or heart, or in other organs such as lung, liver, kidney, or the brain. In particular examples, models of MI are used to assess the activity of the antibody.

Exemplary antagonist antibodies of CD14 effect a decrease in CD14 activity or levels of at least 5%, at least 10%, at least 25%, at least 50%, at least 60%, at least 75%, or at least 85% or more compared to in the absence of the antibody. in some examples, the antibody may result in a decrease in CD14 agonist activity or levels such that the agonist activity or level of CD14 is no longer detectable in the presence of the antibody. Such a decrease may be seen in the sample being tested or, for example where the method is carried out in an animal model.

Preferably, the antibody is a specific antagonist of CD14 as described above. However, this does not mean that a specific antagonist of CD14 has a complete absence of off-target antagonistic activity. In this regard, the specific antagonist of CD14 may have negligible or a minor direct binding and effect on other cellular components, such that the antagonism of the activity, signaling or expression of a non-CD14 cellular component, is less than less than 15%, less than 10%, less than 5%, less than 1%, or less than 0.1% of the direct binding and effect of that agent on the activity, signaling or expression of CD14.

Levels or amounts of CD14 may be measured by assessing expression of the CD14 gene. Gene expression may be assessed by looking at mRNA production or levels or at protein production or levels. Expression products such as mRNA and proteins may be identified or quantified by methods known in the art. Such methods may utilize hybridization to specifically identify the mRNA of interest. For example such methods may involve PCR or real-time PCR approaches. Methods to identify or quantify a protein of interest may involve the use of antibodies that bind that protein. For example, such methods may involve western blotting. Regulation of CD14 gene expression may be compared in the presence and absence of an antibody. Thus, antibodies can be identified that decrease CD14 gene expression compared to the level seen in the absence of the antibody. Such antibodies may be suitable antagonists of CD14 in accordance with the present disclosure.

The methods for identifying suitable antagonist antibodies for use in accordance with the present disclosure may assess the agonist activity of CD14. For example, such a method may be carried out using peripheral blood mononuclear cells. Such cells will produce cytokines such as IL-1α, IL-6, TNF-α, IFN-β, IL-1β, IL-17 and IL-8 on response to stimulation with, for example, LPS. Methods may therefore comprise combining peripheral blood mononuclear cells with the antibody or a vehicle and adding LPS. The cells may then be incubated for an amount of time (e.g., 24 hours) to allow the production of pro-inflammatory mediators such as cytokines. The level of cytokines such as IL-1α, IL-6, TNF-α, IFN-β, IL-1β, IL-17 and IL-8 produced by the cells in that time period can then be assessed. If the antibody has anti-CD14 properties, then the production of such cytokines should be reduced compared to the vehicle-treated cells.

### 3. Ancillary Agents and Interventions

The CD14 antagonist antibody may administered alone or in combination with other active agents (also referred to as “ancillary agents”) or other interventions, such as agents and interventions useful for the treatment of MI.

Ancillary agents suitable for the purposes of the present disclosure include, for example, fibrinolytic agents, beta blockers, high intensity statins (e.g. atorvastatin or rosuvastatin), angiotensin converting enzyme (ACE) inhibitors and platelet inhibitors.

In one example, the ancillary agent is a beta blocker (or beta-adrenoceptor antagonist). Suitable beta-blockers may be non-selective or beta-1 selective. Non-selective agents bind to both beta-1 and beta-2 receptors and induce antagonizing effects via both receptors. Non-limiting examples of non-selective beta blockers include propranolol, carvedilol, sotalol, and labetalol. Beta-1 receptor-selective blockers only bind to the beta-1 receptors and include, for example, atenolol, bisoprolol, metoprolol, and esmolol.

In other examples, the ancillary agent is a fibrinolytic agent, such as, for example, streptokinase, anistreplase or a tissue plasminogen activator (e.g. tenecteplase, reteplase or alteplase).

In further examples, the ancillary agent is a platelet inhibitor, such as aspirin, a P2Y12 inhibitors (e.g. ticlopidine, clopidogrel, ticagrelor or prasugrel) or a glycoprotein IIb/IIIa receptor antagonist.

In another example, the ancillary agent is an ACE inhibitor. Non-limiting examples of ACE inhibitors include benazepril, captopril, enalapril, fosinopril, Lisinopril, moexipril, perindopril, quinapril, Ramipril and trandolapril.

In another example, administration of the antibody is in conjunction with an intervention, such as a percutaneous coronary intervention (PCI; also known as coronary angioplasty) or coronary artery bypass grafting (CABG). Preferably, PCI is performed within 12-24 hours of onset of MI symptoms.

When combination therapy is desired, the CD14 antagonist antibody is administered separately, simultaneously or sequentially with one or more ancillary agents or interventions. In some embodiments, this may be achieved by administering, such as systemically, a single composition or pharmacological formulation that includes both types of agent, or by administering two separate compositions or formulations at the same time, wherein one composition includes the CD14 antagonist antibody and the other the ancillary agent. In other embodiments, the treatment with the CD14 antagonist antibody may precede or follow the treatment with the ancillary agent by intervals ranging from minutes to hours or even days or weeks.

In some situations, the antibody and ancillary agent are administered within about 1-12 hours of each other or within about 2-6 hours of each other. In other situations, it may be desirable to extend the time period for treatment significantly, however, where one or more days (e.g. 1, 2, 3, 4, 5, 6, 7 or 8 days) lapse between the respective administrations. In embodiments where the ancillary agent is administered separately to the CD14 antagonist antibody, it will be understood that the ancillary agent can be administered by a method which is different to that of the administration method used for the CD14 antagonist antibody. In further embodiments, where an intervention (e.g. PCI) is performed on the subject, the antibody is administered to the subject within 72 hours of the PCI, such as at the time of or within 12, 24, 36 or 48 hours of the intervention.

Where two or more agents are administered to a subject “in conjunction” or “concurrently” they may be administered in a single composition at the same time, or in separate compositions at the same time, or in separate compositions separated in time.

### 4. Compositions

As described herein, the use of a CD14 antagonist antibody, whether alone or in combination with ancillary agents, can treat MI. The CD14 antagonist antibody and optionally the ancillary agent can be administered either by themselves or with a pharmaceutically acceptable carrier. Thus, also provided herein are compositions comprising a CD14 antagonist antibody for use in treating MI.

The CD14 antagonist antibodies may be formulated in a conventional manner using one or more pharmaceutically acceptable carriers, stabilizers or excipients (vehicles) to form a pharmaceutical composition as is known in the art, in particular with respect to protein active agents. Carrier(s) are “acceptable” in the sense of being compatible with the other ingredients of the composition and not deleterious to the recipient (e.g. patient) thereof. Suitable carriers typically include physiological saline or ethanol polyols such as glycerol or propylene glycol.

The antibody may be formulated as neutral or salt forms. Pharmaceutically acceptable salts include the acid addition salts (formed with free amino groups) and which are formed with inorganic acids such as hydrochloric or phosphoric acids, or such organic acids such as acetic, oxalic, tartaric and maleic. Salts formed with the free carboxyl groups may also be derived from inorganic bases such as sodium, potassium, ammonium, calcium, or ferric hydroxides, and organic bases as isopropylamine, trimethylamine, 2-ethylamino ethanol, histidine and procaine.

The compositions may be suitably formulated for systemic administration, including intravenous, intramuscular, subcutaneous, or intraperitoneal administration and conveniently comprise sterile aqueous solutions of the antibody, which are preferably isotonic with the blood of the recipient. Such formulations are typically prepared by dissolving solid active ingredient in water containing physiologically compatible substances such as sodium chloride, glycine, and the like, and having a buffered pH compatible with physiological conditions to produce an aqueous solution, and rendering said solution sterile. These may be prepared in unit or multi-dose containers, for example, sealed ampoules or vials.

The compositions may incorporate a stabilizer, such as for example polyethylene glycol, proteins, saccharides (for example trehalose), amino acids, inorganic acids and admixtures thereof. Stabilizers are used in aqueous solutions at the appropriate concentration and pH. The pH of the aqueous solution is adjusted to be within the range of 5.0-9.0, preferably within the range of 6-8. In formulating the antibody, anti-adsorption agent may be used. Other suitable excipients may typically include an antioxidant such as ascorbic acid. The compositions may be formulated as controlled release preparations which may be achieved through the use of polymer to complex or absorb the proteins. Appropriate polymers for controlled release formulations include for example polyester, polyamino acids, polyvinyl, pyrrolidone, ethylenevinylacetate, and methylcellulose. Another possible method for controlled release is to incorporate the antibody into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatin-microcapsules and poly(methylmethacylate) microcapsules, respectively, or in colloidal drug delivery systems, for example, liposomes, albumin microspheres, microemulsions, nanoparticles, and nanocapsules or in macroemulsions.

A CD14 antagonist antibody and optionally an ancillary agent may also be administered directly to the airways in the form of an aerosol. For use as aerosols, the inhibitors of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

One of skill in the art will recognize that formulations are routinely designed according to their intended use, i.e. route of administration.

### 5. Methods of Treatment

The present disclosure provides for therapeutic methods of treating a subject with MI. In some examples, the MI is STEMI. In other examples, the MI is NSTEMI. In further examples, the MI is type 1, type 2, type 3, type 4a, type 4b or type 5 MI.

In some embodiments, the methods of the present disclosure may include an assessment of whether the subject has MI, and in particular NSTEMI or STEMI, and/or type 1, type 2, type 3, type 4a, type 4b or type 5 MI, and therapy proceeds on the basis that they do have MI (optionally of one of the afore-mentioned types).

Contemplated herein are therefore methods for treating MI in a subject by administering to the subject a CD14 antagonist antibody, and optionally administering an ancillary agent or performing an intervention (e.g. PCI). The CD14 antagonist antibody, and optionally the ancillary agent (collectively referred to herein as “therapeutic agents”), will be administered in an “effective amount(s)”, to achieve an intended purpose in a subject, such as the reduction or prevention of one or more symptoms or consequences of MI, e.g. the reduction or prevention of damage to the heart muscle, and/or the reduction or prevention of a loss of heart function (e.g. a reduction or prevention of systolic dysfunction). The dose of therapeutic agents(s) administered to a patient should be sufficient to achieve a beneficial response in the subject. In some examples, the administration of the antibody (optionally with an ancillary agent) results in a reduction of systolic dysfunction (or ventricular dysfunction) compared to when the antibody is not administered (i.e. an increase in systolic function or ventricular function compared to when the antibody is not administered).

The quantity or dose frequency of the therapeutic agent(s) to be administered may depend on the subject to be treated, inclusive of their diagnosis (e.g. the type of MI or the symptoms they present with), age, sex, weight and general health condition thereof. In this regard, precise amounts of the therapeutic agent(s) for administration will depend on the judgment of the practitioner. One skilled in the art would be able, by routine experimentation, to determine an effective, non-toxic amount of a CD14 antagonist antibody, and optionally an ancillary agent described herein, for administration to a subject. In particular examples, the amount of CD14 antagonist antibody administered to a subject is between 0.1 mg/kg and 50 mg/kg, between 0.5 mg/kg and 40 mg/kg, between 2 mg/kg and 20 mg/kg or between 5 mg/kg and 10 mg/kg. In particular examples, the amount of CD14 antagonist antibody administered to a subject is (or is about) 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg/kg.

The CD14 antagonist antibody may be administered to the subject as a single dose or multiple doses. In particular embodiments, the CD14 antagonist antibody is administered as a single dose (e.g. a single bolus injection or a single discrete infusion). In embodiments where the CD14 antagonist antibody is administered as multiple doses, preferably no more than 3 doses are administered, and these are administered within about 6 hours, 12 hours, 18 hours, 24 hours, 36 hours, 48 hours, 60 hours or 72 hours of one another. In particular embodiments, only 1, 2, or 3 doses of the CD14 antagonist antibody is administered.

In some examples, the CD14 antagonist antibody is administered to the subject at any time up to 4 days post-MI or post-MI diagnosis. In one example, the CD14 antagonist antibody is administered to the subject up to 6, 8, 10, 12, 18, 24, 36, 48, 60, 72, 84 or 96 hours post-MI or post-MI diagnosis. For example, the CD14 antagonist antibody may administered to the subject in a single dose up to 6, 8, 10, 12, 18, 24, 36, 48, 60, 72, 84 or 96 hours post-MI or post-MI diagnosis. In another example, the CD14 antagonist antibody is administered to the subject in two or more doses up to 6, 8, 10, 12, 18, 24, 36, 48, 60, 72, 84 or 96 hours post-MI or post-MI diagnosis. For example, the first dose may be administered up to 24 hours post-MI or post-MI diagnosis, and the second dose may be administered a further 24-48 hours later.

In particular examples, the CD14 antagonist antibody is administered to the subject between 2 and 96 hours, between 4 and 96 hours, between 6 and 96 hours, between 2 and 72 hours, between 4 and 72 hours, between 6 and 72 hours, between 2 and 48 hours, between 4 and 48 hours, between 6 and 48 hours, between 2 and 24 hours, between 4 and 24 hours, between 6 and 24 hours, between 2 and 18 hours, between 4 and 18 hours, between 6 and 18 hours, between 2 and 12 hours, between 4 and 12 hours, or between 6 and 12 hours post-MI.

In instances where the subject receives an intervention such as PCI, the CD14 antagonist antibody may be administered at the time of PCI, and/or after PCI, e.g. within 6, 8, 10, 12, 18, 24, 36, 48, 60, 72, 84 or 96 hours of the PCI.

In order that the invention may be readily understood and put into practical effect, particular preferred embodiments will now be described by way of the following non-limiting example.

### EXAMPLES

### Example 1

### Assessment of the Effect of Anti-CD14 Treatment Following STEMI (Study 1)

A study was performed to evaluate the effect of two different dosage protocols of an anti-CD14 antagonist antibody on the mouse heart, 7 d following STEMI. The active agent used in the study was the biG53 F(Ab′)2 antibody, which functionally inhibits PAMP-dependent cytokine production in a dose dependent manner similar to that observed with the anti-human CD14 mAb IC14, currently used in human studies (International patent application no. PCT/US2020/043619). Briefly, a pre-operative echocardiogram was performed just prior to STEMI surgery, which involved 55 minutes of ventricular occlusion by ligation. Reperfusion was performed at 1 hr post-surgery by release of the ligature. Mice were administered a dosage of biG53 F(Ab′)2 at 5 μg/g body weight (i.e. ˜150 μg/30 g mouse) immediately prior to reperfusion (intravenous (i.v.) dose) and/or at 24 hours post-surgery (intraperitoneal (i.p) dose), such that a single dose or dual dose was administered. The following groups of mice were included in the study:
Control: I/R, vehicle I.V. at 1 h+vehicle I.P. at 24 h (n=8)
Single dose: I/R, anti-CD14 I.V. at 1 h, vehicle I.P. at 24 h (n=8)
Dual dose: I/R, anti-CD14 I.V. at 1 h+anti-CD14 I.P. at 24 h (n=8)

The primary endpoint for this study was echocardiographic examination of systolic function 7 days following STEMI surgery. Circulating pro-inflammatory markers and histology of myocardial fibrosis and CD68+ cell infiltration was also investigated.

### A. Methods

### Randomisation and Blinding

The study was a randomised, blinded study.

### Myocardial Infarction Surgery

A total of 33 male wild-type C57B16 mice (2 batches, n=15 and n=18, respectively) received ischemia-reperfusion surgery at 8.5-9.5 WOA. Briefly, mice were anaesthetized with a ketamine (80-100 mg/kg), xylazine (10-20 mg/kg) and atropine (1-2 mg/kg) mixture before surgical site shaving and intubation. Mice were ventilated (0.2-0.3 ml at 100-140 breaths per minute) on a sterile heated pad and the surgical site (left chest) prepared with and sterilised bupivicaine (2 mg/kg) was administered intra-dermally before a left thoracotomy was performed with sterile instruments. 7-0 sterile suture was used to ligate the left anterior descending coronary artery, ˜2 mm below the left atrial appendage, and reversibly tied using sterile loops, exteriorised during surgical closure (internal intercostal, external skin) with 6-0 prolene suture.

Mice were transferred to a heated vital sign monitoring station where electrocardiography (ECG) and rectal temperature were observed while ventilation was maintained. Mice were then extubated upon resumption of spontaneous breathing and returned to a recovery cage (half of base area was heated to encourage movement through behavioural autoregulation of temperature. Following 55 minutes of occlusion of the left anterior descending coronary artery, recovering mice received IV. injection of biG53 F(Ab′)2 or vehicle, immediately prior to reperfusion (ligation release) at 1 h.

Two mice died before recovery from surgery. All remaining mice were monitored 2-3 times per day for the first 5 days following surgery. All remaining mice recovered fully (resumed normal behaviour, baseline scores of pain/discomfort monitoring) within 24 h (n=31, 94% of operated mice).

### STEMI Model Screening by Echocardiography at 24 h: Relative Tissue Displacement

At 24 h post-surgery, all 31 remaining mice received echocardiography to assess area-at-risk. Briefly, mice were anaesthetised with isoflurane (induction: 3-4.5% in room air, maintenance: 1-2% in room air) and placed on a heated and articulated ECG platform. Gated (EKG) and ungated parasternal long-axis cine loops were obtained by an ultra-high frequency ultrasound probe (MS-550D) using the Vevo® 2100 System (Visualsonics, Fujifilm, Canada). All mice recovered as expected. Analysis was performed using the manufacturer's VevoLAB software to discern inactive from active relative radial tissue displacement in the long axis. Inactive/zero relative tissue displacement provided a rigorous surrogate for ischemic area/infarct size and was used to exclude mice with small/irregular infarctions (resulting from e.g. missed ligation or collateral branching of the coronary arteries).

Only mice with tissue displacement 45±10% of the left ventricle were included in the study (n=13, batch 1; n=13, batch 2). A total of 26 (84%) surgically recovered mice were included in the study for all assessments (Group A: n=10; Group B, n=8; Group C: n=8). Additional predetermined exclusion criteria were applied to this project (i.e. data with technical insufficiency) however no additional data required exclusion.

### Echocardiographic Analysis of Systolic Function at 7d

Left ventricular echocardiography imaging was performed to obtain left parasternal long-axis loops as described above. Left ventricular end-diastolic and end-systolic areas were traced at the endocardial border. From these areas, end-diastolic, end-systolic and stroke volumes; cardiac output and ejection fraction were calculated based on formulae within the software (VevoLAB 3.2.5, Visualsonics, Fujifilm, Canada).

### Autopsy at 7d

Following echocardiography at 7 d post-surgery, mice were anaesthetised with ketamine, xylazine and atropine before cardiac puncture and secondary euthanasia was performed (cervical dislocation). A mean of 1.1±0.1 ml whole blood was collected from each mouse, and a comprehensive autopsy was performed.

### Tissue Collection (at 7d) and Histology

Whole hearts were excised and photographed using a surgical microscope (ZEISS OPMI Pico, Carl Zeiss Meditec AG, Germany) before the four chambers were dissected. The length of the left ventricle (LV) was measured and transversely sectioned at its longitudinal midpoint for fixation in 10% neutral buffered formalin.

Once fixed for 48-72 h, each LV was embedded in paraffin wax and sectioned. Briefly, blocks were trimmed to full facing tissue and 5×4 μm sections were collected across 5× slides. Blocks were trimmed again by 250 μm and 5×4 μm sections were collected alongside the first sections (across the same 5 slides). This 250 μm trimming and 5×4 μm sectioning was repeated until tissue was exhausted or 5 sections were collected across each slide.

Masson's Trichrome staining was performed on one slide from each LV before bright-field scanning. Immunofluorescent staining was performed on another slide from each LV, using antibodies for CD68 (Abcam, Ab125212), Troponin I (Invitrogen, MA5-12960) and DAPI before dark-field scanning. All bright-field scanning was performed using identical settings for each LV and all dark-field scanning was performed using identical settings for each LV.

Analysis of Masson's Trichrome and Immunofluorescence imagery was performed using an automated (macro) approach. Briefly, for Masson's Trichrome analysis, anatomically equivalent mid-ventricular sections (at level of the papillary muscle) were analysed by separating red and blue channels and delineating blue-only area (positive) from red/blue area (negative). Thresholds for positivity were set at 0-100 or 0-130 and total tissue threshold was set to 0-230.

For immunofluorescence (CD68+ cell) analysis, cellular water-shedding was performed using DAPI-stained nuclei (channel 1) and analysing co-localisation of CD68 antibody (channel 2) with a mean intensity threshold of 0-750 and troponin T (channel 3) total tissue threshold of 150

### B. Results

### Acute Assessments of Myocardial Infarction: First 24 h Post-Ischemia

### Post-Surgical Electrocardiography

All 26 mice included in this pilot study were confirmed to have ST-elevation/disruption following myocardial infarction surgery. ECG recordings were noted to be similar in morphology between each group.

### Echocardiographic Assessment of Relative Wall Displacement

Successful infarction in each heart (left ventricle) was reconfirmed by echocardiographic assessment of relative tissue displacement at 24 h. Analysis of relative tissue displacement showed no differences between the three groups of mice (44±5, 47±4, 44±4, respectively; mean±SD, p>0.05).

### Sub-Acute Assessments of Myocardial Infarction: 7 d Post-Ischemia

### Echocardiographic Assessment of Left Ventricular Volumes and Function

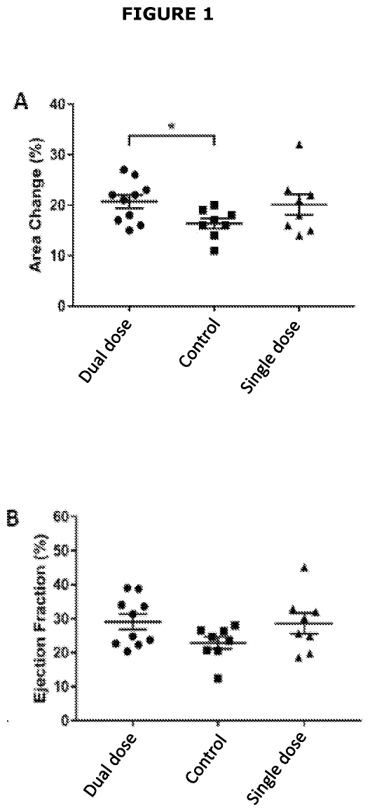

Heart rates were similar between groups at the time of echocardiography, 7 days after STEMI surgery (Table 1, p>0.05). The Dual Dose group had improved systolic function as assessed by echocardiographic left ventricular area change (LVAC; 21±4 vs. 16±3% in Controls, p<0.05) and longitudinal fractional shortening (8.3±1.4 vs. 6.4±1.1% in Controls, p<0.05) (FIG. 1 and Table 1). A non-significant, otherwise intermediate change was observed in the Single Dose group (p>0.05).

An associated, yet non-significant absolute increase of 6% ejection fraction was observed in the Dual Dose group (29±7 vs. 23±5 in Controls, p>0.05), a trend of ˜25% relative increase in systolic function, corresponding with the significant ˜30% relative improvement observed in LVAC.

TABLE 1

| | Control | Single dose | Dual dose |
|---|---|---|---|
| n | 8 | 8 | 10 |
| Echocardiography | | | |
| Heart rate (BPM) | 489 ± 32 | 503 ± 38 | 504 ± 34 |
| 2-dimensional Measurements | | | |
| End-diastolic area (mm2) | 30 ± 4 | 28 ± 5 | 28 ± 5 |
| End-systolic area (mm2) | 25 ± 4 | 23 ± 5 | 22 ± 5 |
| Area change (%) | 16 ± 3 | 20 ± 6 | 21 ± 4* |
| Longitudinal fractional shortening (%) | 6.4 ± 1.1 | 7.9 ± 1.9 | 8.3 ± 1.4* |
| 3-dimensional Calculations | | | |
| End-diastolic volume (ul) | 101 ± 25 | 87 ± 25 | 84 ± 23 |
| End-systolic volume (ul) | 78 ± 22 | 63 ± 23 | 61 ± 21 |
| Stroke Volume (ul) | 23 ± 6 | 24 ± 6 | 23 ± 4 |
| Cardiac output (ul/min) | 11 ± 3 | 12 ± 3 | 12 ± 2 |
| Ejection fraction (%) | 23 ± 5 | 29 ± 9 | 29 ± 7 |

*p < 0.05 between control and dual dose group.
NSD—No significant differences

### Autopsy Biometrics

All organ gravimetric parameters were observed to be similar between groups at autopsy (Table 2). One mouse in the control group, but not in either treatment group, was revealed to have atrial thrombus at autopsy; typically observed in heart failure in mice models of STEMI.

### Serum Analysis

A representative sample from each group was analysed by multiplex assay (R & D Systems, Mulitplex Tool) to establish analyte ranges for subsequent ELISA analysis. The multiplex assay returned all values lesser than the detection limit (not detected; N.D.). High sensitivity ELISA kits for TNF-alpha and IL-1beta (Invitrogen, 88-7013-22) were subsequently used, with standard curves extended to a lower detection limit of ˜2 pg/mi (manufacturer's recommended limit 8 pg/ml). Despite the high sensitivity ELISA range and successful establishment of standard curves for interpolation, all sample values for both analytes were below the detectable ranges.

### Histology

Masson's trichrome brightfield imaging of the mid-ventricle of mice from each group was performed to visualize interstitial and patch fibrosis, and to discern this area from non-fibrotic tissue, so as to provide a semi-quantitative analysis of fibrosis area (as a percentage of total area). While fibrosis was observed in each group, there was no statistical difference in the percentage of fibrosis between groups (Table 2).

TABLE 2

| | Control | Single dose | Dual dose | Difference |
|---|---|---|---|---|
| n | 8 | 8 | 10 | |
| Total (All 10 | | | | |
| sections) | | | | |
| Total area (mm²) | 40.9 ± 7.9 | 39.6 ± 6.3 | 41.5 ± 5.6 | NSD |
| Positive area | 6.5 ± 1.4 | 5.5 ± 2.1 | 6.5 ± 2.2 | NSD |
| (mm²) | | | | |
| Fibrosis (% total | 16 ± 2 | 14 ± 3 | 16 ± 4 | NSD |
| area) | | | | |
| Mid-ventricular | | | | |
| Section (Single | | | | |
| section) | | | | |
| Total area (mm²) | 4.3 ± 1.0 | 3.9 ± 1.0 | 3.7 ± 0.5 | NSD |
| Positive area | 0.7 ± 0.3 | 0.8 ± 0.3 | 0.7 ± 0.1 | NSD |
| (mm²) | | | | |
| Fibrosis (% total | 17 ± 3 | 19 ± 5 | 20 ± 4 | NSD |
| area) | | | | |

Mean ± SD.
NSD—No significant differences

Immunofluorescent imaging of mid-ventricular sections was performed to visualize CD68+ cells and provide a semi-quantitative analysis of CD68+ cells as a percentage of total cells. While significant peri-infarct CD68+ cellular infiltration was observed in the myocardium, there was no difference between the groups (Table 3).

TABLE 3

| | Control | Single dose | Dual dose | Difference |
|---|---|---|---|---|
| n | 8 | 10 | 10 | |
| Total (All | | | | |
| sections) | | | | |
| Total cells (×10⁶) | 5.1 ± 0.7 | 5.1 ± 0.5 | 5.5 ± 0.7 | NSD |
| CD68+ cells | 0.6 ± 0.2 | 0.7 ± 0.1 | 0.6 ± 0.2 | NSD |
| (×10⁶) | | | | |
| CD68+ cell | 12 ± 3 | 13 ± 2 | 12 ± 3 | NSD |
| percentage (% | | | | |
| total cells) | | | | |
| Mid-ventricular | | | | |
| Section (Single | | | | |
| section) | | | | |
| Total cells (×10⁶) | 0.6 ± 0.1 | 0.6 ± 0.1 | 0.5 ± 0.1 | NSD |
| CD68+ cells | 0.1 ± 0.0 | 0.1 ± 0.0 | 0.1 ± 0.0 | NSD |
| (×10⁶) | | | | |
| CD68+ cell | 15 ± 5 | 19 ± 3 | 17 ± 5 | NSD |
| percentage (% | | | | |
| total cells) | | | | |

Mean ± SD.
NSD—No significant differences

### C. Discussion

STEMI with subsequent percutaneous coronary intervention (reperfusion) induces excessive cardiac inflammation and loss of working heart muscle cells in the acute/sub-acute phase. This evokes a progressive process of fibrosis and cardiac remodelling leading to the development of heart failure.

Throughout the acute and sub-acute phases of STEMI, immense bi-phasic inflammatory responses drive disease followed by repair. Research to date has been focused on suppressing this inflammation with “blunt” pharmacological agents. However, these anti-inflammatories can disrupt reparative processes by non-selectively suppressing all activities of leukocytes, including those involved in damage resolution and tissue repair. Thus, interventions which selectively inhibit the damaging (whilst sparing the reparative anti-inflammatory) cells and processes may reduce acute damage as well as the degree of cardiac remodelling and loss of function associated with post-STEMI heart failure.

Damage-Associated Molecular Pattern (DAMP) molecules are released by damaged cardiomyocytes during acute STEMI and cause resident pro-inflammatory macrophages to attract circulating leukocytes (primarily neutrophils) from the blood. Following phagocytosis of the damaged and necrotic cells, these neutrophils undergo apoptosis, which promotes the resolution-phase of tissue repair. CD14 is an important cofactor for a number of pattern recognition receptors which promote DAMP-driven inflammation in a variety of cell types. Inhibition of CD14 reduces pro-inflammatory but not anti-inflammatory cytokines.

The present study was performed to determine whether the actions of CD14 may make it a good therapeutic target in the acute setting of STEMI-associated inflammation. It was hypothesized that the short-term inhibition of CD14 could reduce the excessive inflammation associated with myocardial infarction and mitigate subsequent damage, fibrosis and remodelling in the murine heart. The study demonstrated that targeting CD14 did indeed have a therapeutic effect in the context of MI.

Echocardiography—The significant ˜30% relative improvements in both left ventricular area change and longitudinal fractional shortening with Dual Dose treatment of anti-CD14 was observed with high confidence. All acquisitions and analyses were performed blinded, and replicate interobserver correlations were of an industry-leading standard (slope=1.0-1.1, r=0.94). Additionally, end-diastolic correlation with whole heart weight further supported the confidence of these findings (r=0.9).

Serum analyses & Histology—Neither pro-inflammatory cytokines were detected by either multiplex or high sensitivity ELISA assay at the 7-day endpoint. This is most likely associated with the temporally phasic acute infiltration of proteolytic macrophages with M1-like characteristics, and concomitant release of pro-inflammatory cytokines in the first 1-3 days following myocardial infarction in mice, which reduces bluntly during the sub-acute “resolution phase” 4-14 days post-myocardial infarction.

The observed prominent infiltrations of CD68+ cells in the myocardium of infarcted mouse hearts at 7 days, in the absence of circulating pro-inflammatory biomarkers, suggests that these macrophages have predominantly M2-like characteristics and are involved in the resolution of the damaged myocardium. This also suggests that the Dual Dose protocol of anti-CD14 treatment does not inhibit the infiltration of M2-like macrophages.

Overall, this study provides the first in vivo evidence of sub-acute cardioprotection following STEMI in mice treated with anti-CD14 antibody. This cardioprotective effect (preserved systolic function at 7 days) was observed most clearly in the dual dose group (5 μg/g anti-CD14 antibody given at both reperfusion and 24 h post-reperfusion), although was observed to a lesser extent in the single dose group.

### Example 2

### Assessment of the Effect of CD14 in M1/M2 Differentiation

The previous study indicated that anti-CD14 treatment does not inhibit the infiltration of M2-like macrophages following MI. To further assess the effect that targeting CD14 has on M1 differentiation, a study was performed to evaluate the ability of IC14, a clinical-grade antibody specific for human CD14, to block differentiation along the M1 pathway using iPSC derived macrophages.

### A. Materials and Methods

Induced pluripotent stem cells (iPSC) from healthy donors were obtained from the iPSC core at the Board of Governors Regenerative Medicine Institute, Cedars-Sinai Medical Center. iPSC were differentiated to M0 macrophages according to the protocol of Yanagimachi et al. (PLoS One, 2013 8, 1-9) Briefly, iPSCs were treated with 5 sequential culture steps over 36 days, with an initial induction of primitive streak-like cells using BMP4, then generation of KDR+CD34+ hemangioblast-like cells using VEGF, SCF and basic FGF, the subsequent generation of hematopoietic cells using hematopoietic cytokines, their differentiation into the monocytic lineage with Flt-3 ligand, GM-CSF and M-CSF and finally their differentiation into M0 macrophages using M-CSF, IFN-γ and IL-4.

iPSCs were differentiated along the M1 lineage by plating derived M0 cells at a concentration of 50,000 cells per well in a 96 well plate and cultured in 200 μl of RPMI 1640 with 10% FBS in the presence of 2 ng/ml IFNγ (eBioscience) and 1 ng/ml LPS (Sigma). After 60 minutes cultures were treated with either IC14 (Implicit Bioscience) or human IgG4 control antibody (Biolegend) over a range of 0.01-1 ug/ml. After an additional 3 hours cells were harvested for RNA extraction using Trizol reagent followed by Direct-zol RNA MiniPrep Kit (Zymo Research). Quantitative RTPCR (qRT-PCR) experiments were performed using a One-Step RT-PCR kit with SYBR Green and run using a Bio-Rad iQ5 Multicolor Real-Time PCR Detection Systems,

### B. Results

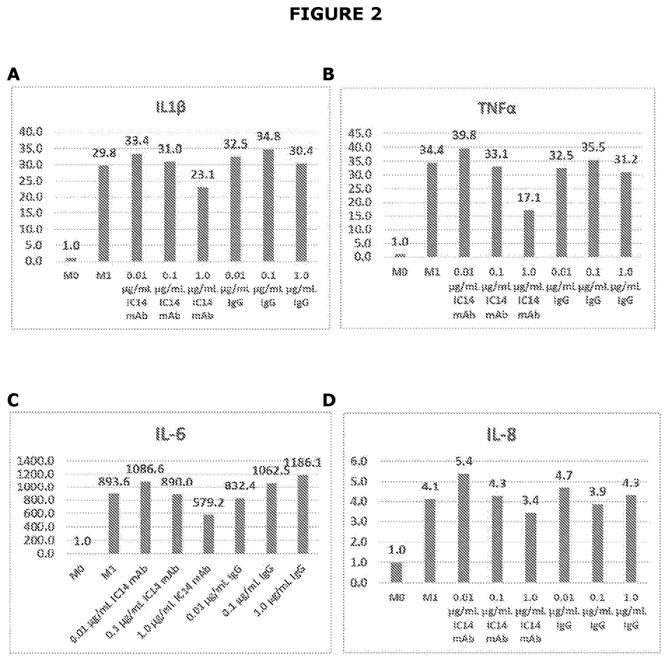

iPSC which have been differentiated to M0 cells can be further induced to M1 macrophages by treatment with LPS and IFNγ. Upon induction, M1 macrophages express IL-1β, TNFα, IL-6 and IL-8 transcripts (FIG. 2). LPS and IFNβ stimulation led to a rapid increase in levels of these transcripts as measured at 4 hours post stimulation. This study showed that M1 differentiation has a CD14-dependent component; the inclusion of IC14 in these cultures at 1 hr post-stimulation resulted in a decrease in the production of IL-1β, TNFα, IL-6 and IL-8. This inhibition was not observed with an isotype control antibody, indicating that it occurred as a direct consequence of blockade of mCD14 with IC14 (FIG. 2).

### C. Discussion

Macrophages play an important role in both initiating and resolving inflammatory processes, functioning in pro- and anti-inflammatory roles. These distinct and opposing processes led to the proposal that macrophages could be assigned to one of two phenotypes; the classically activated (inflammatory) macrophage (designated M1) or the alternatively activated (or wound-healing) macrophage (M2). This initial classification into two opposing functional states is likely overly simplistic, failing to reflect the complex nature of the states themselves and the plasticity of the polarization process. Rather it is likely more appropriate to consider macrophage polarization as a continuum of functional states. It is now accepted that macrophage polarization is a multifactorial process in which multiple factors interact to generate distinct activation states. These activation states are themselves plastic and can be modified in response to changing environmental influences.

Alterations in the balance between M1 and M2 phenotypes are associated with a number of diseases. For example, in cancer the presence of M2 macrophages within tumors and a decreased M1/M2 ratio is associated with a poor prognosis. In contrast inflammatory and autoimmune diseases are associated with an increased M1/M2 ratio.

Although macrophage polarization in vivo is regarded as a multifactorial process, M1 differentiation can be reproduced in vitro by stimulation with IFNγ and LPS, two stimuli which replicate the activation found at sites of inflammation by cytokines and TLR agonists. In this study, the ability of IC14 to block differentiation along the M1 pathway using iPSC derived macrophages was assessed. This study indicated that IC14 can decrease the production of all four key inflammatory mediators, TNFα, IL-1β, IL-6 and IL-8, during the process of M1 differentiation. Blocking the development of M1 macrophages or promoting their polarization along an alternative protective (M2) pathway may protect from pathologic inflammation following MI.

### Example 3

### Assessment of the Effect of Anti-CD14 Treatment Following STEMI (Follow-Up Study)

A follow-up study was performed to evaluate the effect of two further dosage protocols of an anti-CD14 antagonist antibody on the mouse heart, 7 d following STEMI. The anti-CD14 antagonist antibody used in the study was the biG53 anti-mouse anti-CD14 mAb (i.e. the full antibody form of the biG53 F(Ab′)2 utilized in Example 1). This mouse antibody is a representative surrogate for the clinical antibody (IC14) described in Example 2. Briefly, a pre-operative echocardiogram was performed just prior to STEMI surgery, which involved 55 minutes of ventricular occlusion by ligation. Reperfusion was performed at 1 hr post-surgery by release of the ligature. Mice were administered a dosage of biG53 mAb at 7 μg/g body weight immediately prior to reperfusion (intravenous (i.v.) dose) and/or at 8-12 hours post-surgery (intraperitoneal i.p dose), and/or 24 hours post-surgery (intraperitoneal (i.p) dose), such that a dual dose or triple dose was administered. The following groups of mice were included in the study:
Group 1. I/R, vehicle I.V. at 1 h+vehicle I.P. at 8-12 h+vehicle I.P. at 24 h (saline control)
Group 2. I/R, 1× isotype I.V. at 1 h+vehicle I.P. at 8-12 h+Isotype I.P. at 24 h (2×7 mg/kg dose) (isotype control)
Group 3. I/R, 1× anti-CD14 I.V. at 1 h+vehicle I.P at 8-12 h+anti-CD14 I.P. at 24 h (2×7 mg/kg dose)
Group 4. I/R, 1×anti-CD14 I.V. at 1 h+anti-CD14 I.P. at 8-12 h+anti-CD14 I.P. at 24 h (3×7 mg/kg dose)

The primary endpoint for this study was echocardiographic examination of systolic function 7 days following STEMI surgery. Serum pro-inflammatory markers (cytokines) and histology of myocardial fibrosis and CD68+ cell infiltration, as well as cardiac cathaterisation haemodynamic measurements, were also investigated at day 7.

### A. Methods

### Randomisation and Blinding

The study was a randomised, blinded study.

Model of ST-Segment Elevation Myocardial Infarction (MI) with Reperfusion

Left anterior descending coronary artery ligation to induce 1 h ischaemia was performed, followed by reperfusion resulting in ST-elevated Myocardial Infarction.

### Electrocardiogram (ECG)

3-lead ECG leads were placed under the skin to record up to 5 mins of ECG tracing to confirm ST elevation immediately post-MI, and during endpoint catheterisation.

### Echocardiography (24 h, Day 7 (D7) Post-MI)

Mice were anaesthetised with isoflurane (4.0% induction, 1.6-1.8% maintenance) and comprehensive echocardiography studies of left ventricular (LV) systolic function were performed using the Vevo2100 systems (Visualsonics, Fujifilm). 24 h echocardiography was analysed to confirm ischemic area homogeneity by our platform-validated tissue displacement mapping technique, an emerging gold standard for the screening of MI model homogeneity. All analysis of ultra-high-frequency parasternal long-axis loops (gated-EKV for 24 h wall displacement mapping) was performed offline and validated.

### Blood Sampling and Analysis (D7 Post-MI)

A cardiac puncture was performed (for blood collection) and processing for serum analyses by commercial Multiplex immunoassay kits (Bio-Plex Pro, Bio-Rad Laboratories, Inc.).

### Autopsy and Tissue Collection (D7 Post-MI)

Comprehensive autopsies were performed for all mice and included weights of heart chambers, lungs, kidney, liver and spleen. Once cardiac dissection was performed, a mid-ventricular transverse ring of the left ventricle was committed to histology, and apical/infarcted ventricle stored for future investigations at −20° C.

### Left Ventricle Histology (D7 Post-MI)

Complex sectioning was performed to produce replicate slides of left (mid-) ventricular sections. Tissues were processed, embedded, stained, imaged and analysed by blinded personnel. Staining protocols included Hematoxylin & Eosin (H & E), Masson's Trichrome, Picrosirius Red and immunofluorescence using antibodies for CD68, Troponin T and DAPI.

### Cardiac Catheterisation and Haemodynamic Assessment (D7 Post-MI)

Mice were anaesthetised with isoflurane (4.0% induction, 1.6-1.8% maintenance) and an intracardiac catheter was passed via the right carotid artery into the ascending aorta to measure arterial pressures, before being advanced into the left ventricle to measure left ventricular pressures and conductance. End-systolic and end-diastolic pressure-volume relationships were observed by compressing the abdominal aorta through the sub-hepatic space. Parallel conductance was corrected for using hypertonic saline infusion into the right jugular vein (5-10 μl) prior to cardiac puncture. Blood was then used to construct a conductance standard curve in calibration cuvette wells of known volumes. Comprehensive haemodynamic analysis was performed offline and validated.

### Statistical Analyses

All data were analysed using GraphPad Prism (V7.0) using one-way ANOVA with Tukey multiple comparisons post-hoc tests. Homogeneity of variance was assessed for all reported parameters using Brown-Forsythe testing, and Kruskal-Wallis (non-parametric) testing used where appropriate. All data are presented as mean±SD in text/tables, and as mean±SEM in figures for comparison.

### Exclusion Criteria

Factors associated with surgical (model) or endpoint technical insufficiency were prospectively defined as the only grounds for exclusion:
Animal: Lack of ST-elevation observed (immediately post-MI surgery) and/or Relative Negative Wall Displacement <35 or >55% (24 h echo)
Endpoint: Technically insufficient imaging/recording for analysis e.g. unsuccessful catheter insertion, unsuccessful histology sectioning/staining

Mice which did not survive surgery to reperfusion (treatment) were also excluded from analysis. Note: All deaths (n=9) occurred before reperfusion i.e. no animals died prematurely after treatment.

### B. Results

### Unblinding

The groups were unblinded following data collection and analysis. The groups were identified as follows:
A. Isotype control
B. 3×dose anti-CD14 Ab
C. Saline control
D. 2×dose anti-CD14 Ab

### Assessments of Ischemic Injury: First 24 h Post-Surgery

### Post-Surgical Electrocardiogram

All 60 mice (15 per group) included in this study were confirmed to have ST-elevation following myocardial infarction surgery.

### Echocardiographic Assessment of Relative Wall Displacement

Successful transmural infarction in each heart (left ventricle) was reconfirmed by echocardiographic assessment of negative relative wall displacement at 24 h. Mice with negative displacement <35 or >55% were excluded from the study. No differences were observed between groups.

### Echocardiographic Assessment of Left Ventricular Volumes and Function 7 Days Post-Surgery

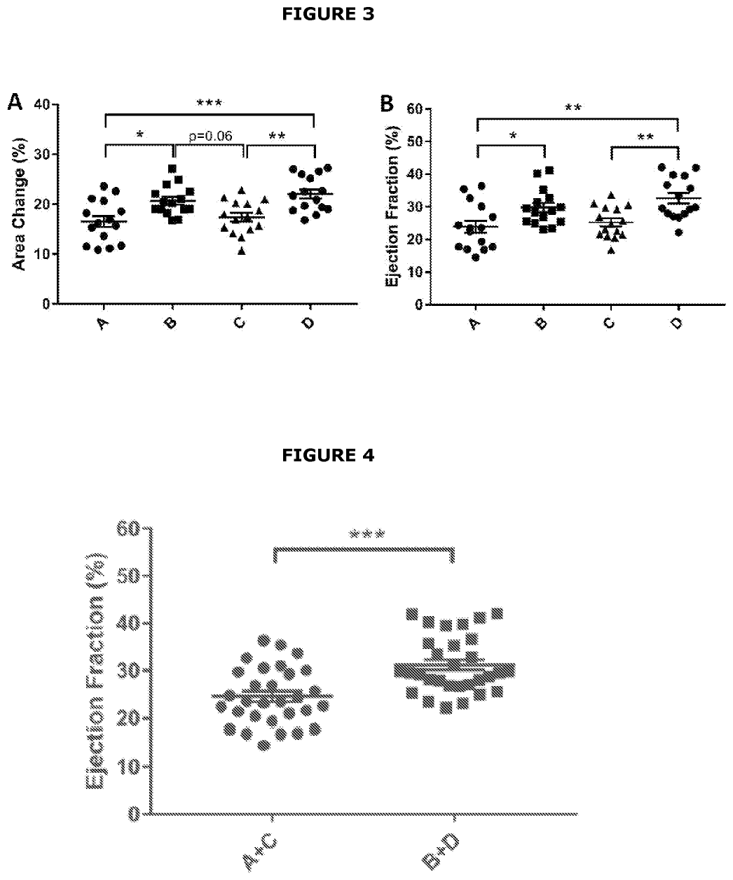

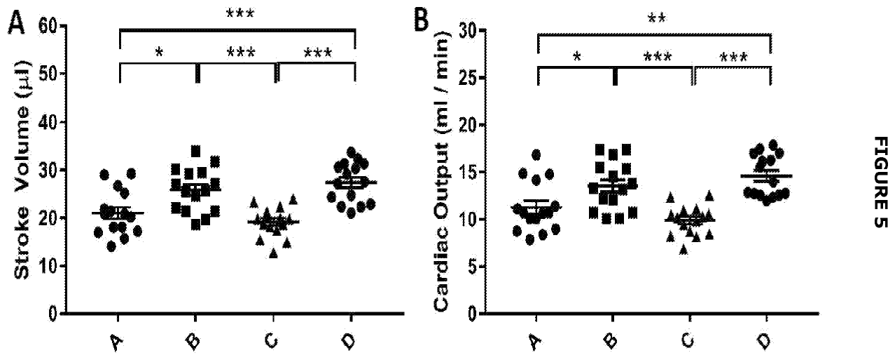

Echocardiographic abnormalities were observed in one mouse at baseline and did not proceed to surgery. Heart rates were similar between groups at the time of endpoint echocardiography 7 days after STEMI surgery (data not shown, ANOVA p=0.371). Left ventricular areas at diastole and systole were measured by tracing endocardial borders in the parasternal long axis. Volumetric values were calculated based on bi-planar assumptions of left ventricular morphology. Differences between mice administered the anti-CD14 antibody and the control mice were observed in longitudinal fractional shortening (data not shown); LV area change (which reflects contractile function of the heart) and ejection fraction (which reflects the percentage of blood pumped out of the left ventricle) (FIGS. 3 and 4). This confirmed the results of the study described in Example 1, with both studies showing an approximate 25% increase in ejection fraction in mice that received the anti-CD14 Ab. Increased stroke volume (which reflects the volume of blood ejected with each heart beat) and cardiac output (which reflects the volume of blood pumped each minute) was also observed in the mice that received the anti-CD14 Ab (FIG. 5).

### Haemodynamic Assessment of Left Ventricular and Arterial Pressures at 7 Days Post-Surgery

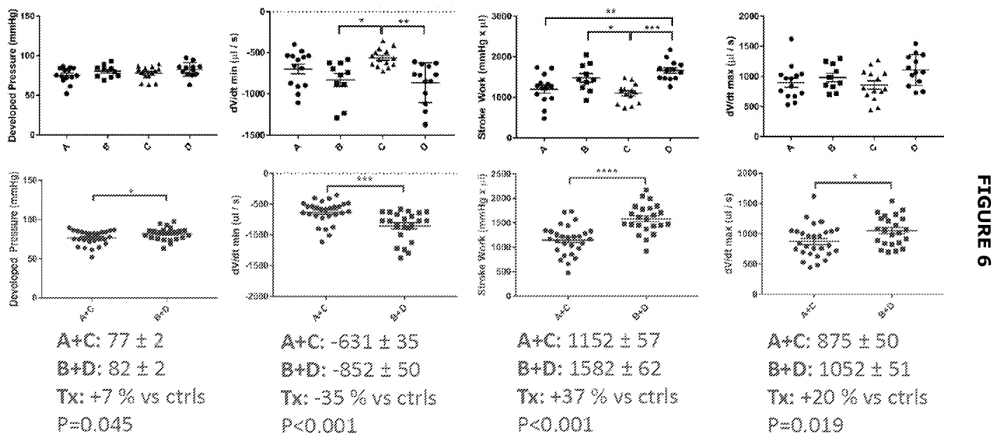

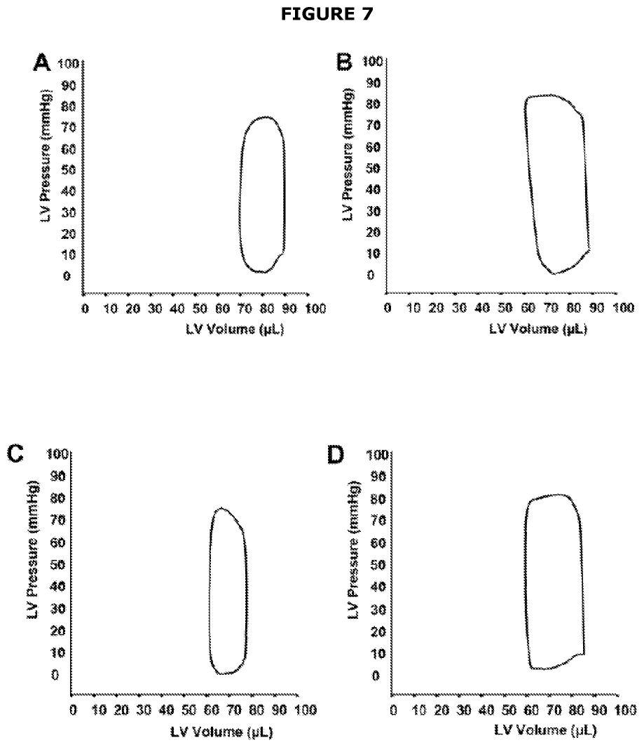

Heart rates were similar between groups at the time of haemodynamic assessment by catheter, 7 days after STEMI (immediately following echocardiography procedures, data not shown, ANOVA p=0.989). As shown in FIGS. 6 and 7, differences between the control groups and the groups administered the anti-CD14 antibody were observed for change in LV volume over time (dV/dt min; reflecting the peak left ventricular ejection rate during contraction), dV/dt max (which reflects the peak LV filling rate during relaxation) and arterial elastance (Ea) (data not shown); and stroke work (which is a function of the mean aortic pressure×stroke volume), indicating more efficient functioning of the heart following STEMI in mice that received the anti-CD14 Ab. No differences were observed for arterial diastolic, systolic or pulse pressures (data not shown).

### Biometrics at Surgery (D0) and 7 Days Post-Surgery

All groups were similar in age at time of surgery (D0) and had similar tibia lengths at endpoint (D7). Body weight at surgery was 4-6% less in group C (saline control group), compared to groups B and D (anti-CD14 Ab groups; data not shown). This small but statistically significant difference was also observed at endpoint (D7). Group D was the only group to significantly increase in body weight 7 days post-surgery (1.9±2.3% vs D0, P<0.01).

The isotype control and anti-CD14 Ab groups (group A, and groups B and D) were observed to have similar organ weights at autopsy. Group C was observed to have smaller heart, left/right ventricle and kidney weights compared to groups B and/or D. For all size/volume parameters measured at D7 adjusted for body weight at surgery.

### Histology at 7 Days Post-Surgery

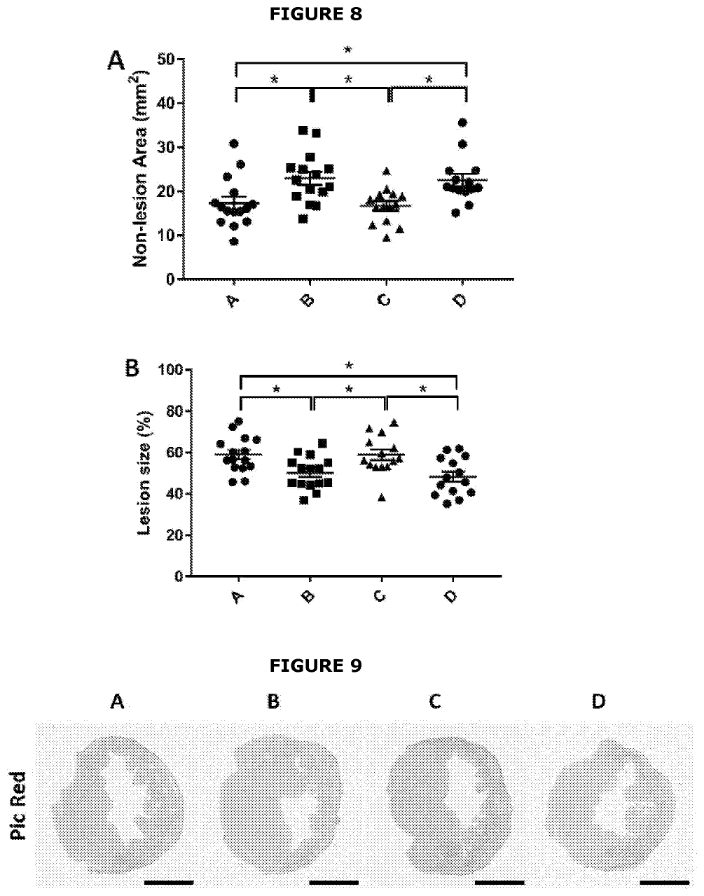

Brightfield Histology: Hearts dissected at autopsy were sectioned at the mid-ventricle. For analysis, lesion (scar, including free wall), and non-lesion (remote tissue, including interventricular septum) areas were assessed using Picrosirius Red-stained sections to quantify the total section, lesion and non-lesion positivity. Differences in total section positivity were observed between groups C (saline control) and D (2× Dose anti-CD14 Ab). Non-lesion area and lesion size (indicative of infarct size) were observed to be different between groups A and C (control groups) vs B and D (anti-CD14 Ab groups) (FIGS. 8 and 9). This histological analysis clearly demonstrated a reduction in fibrosis in the hearts of mice that received anti-CD14 Ab compared to the control mice (FIG. 9; where dark grey represents collagen (stained red) and light grey represents myocardium (stained yellow).

### Immunofluorescence Histology

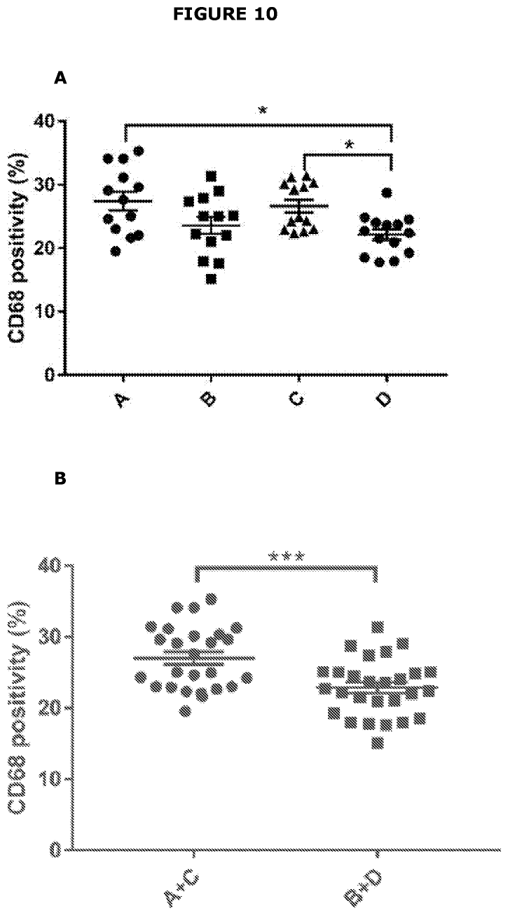

Differences were not observed between total cell count or CD68+ cell counts in whole left mid-ventricular sections (data not shown). However, differences were observed between groups A and C (control groups) versus group D (2× Dose anti-CD14 Ab) in CD68 positivity (CD68+ cell count as a percentage of total cell count, with the mice that received anti-CD14 Ab showing reduced CD68 positivity (FIG. 10).

### Serum Analyses at 7 Days Post-Surgery

A randomised subset of samples (n=10) from each group was analysed by multiplex (Bio-Plex Pro Mouse Cytokine Mulitplex Assay and Bio-Plex Pro TGF-β 3-plex Assay, Bio-Rad Laboratories). Table 4 shows the serum analysis results at 7 days post-surgery: TNF-α—tumour necrosis factor alpha, IL—interleukin, MCP—monocyte chemoattractant protein, TGF—tumour growth factor. §—values fall beyond detection limits recommended by the manufacturer. F—failed homogeneity of variance testing, non-parametric analysis used. ND—None detected. Mean±SD. No significant differences were observed between groups for any analyte. This is likely due to the relatively late time point, with differences more likely to be seen within the first few days of MI and more localized to the heart tissue itself rather than systemic circulation.

TABLE 4

| | Detect. Range | A | B | C | D | ANOVA |
|---|---|---|---|---|---|---|
| n | | 10 | 10 | 10 | 10 | |
| Serum analyses | | | | | | |
| TNF-α (pg/ml) | 0.81-590 | 0.5 ± 0.1^{§} | 0.5 ± 0.1^{§} | 0.4 ± 0.1^{§} | 0.4 ± 0.1^{§} | P = 0.788 |
| IL-6 (pg/ml) | 8.4-6,130 | 3.5 ± 4.8^{§} | 8.3 ± 6.1^{§} | 7.0 ± 5.5^{§} | 3.4 ± 2.5^{§} | P = 0.159 |
| IL-10 (pg/ml) | 4.6-3,350 | 1.5 ± 0.4^{§} | 1.3 ± 0.6^{§} | 1.2 ± 0.3^{§} | 1.2 ± 0.3^{§} | P = 0.242 |
| IL-1beta (pg/ml) | 76.7-55,940 | 7.6 ± 5.3^{§} | 12.2 ± 5.4^{§} | 13.9 ± 4.9^{§} | 10.8 ± 7.2^{§} | P = 0.166 |
| MCP-1 (pg/ml) | 86-62,940 | 54.2 ± 8.2^{§} | 58.4 ± 15.9^{§} | 55.5 ± 6.6^{§} | 52.5 ± 13.2^{§} | P = 0.717 |
| TGF-β1 (ng/ml) | 0.001-12.577 | 1.2 ± 1.5 | 1.5 ± 1.3 | 1.1 ± 0.4 | 1.1 ± 0.5 | F, P = 0.565 |
| TGF-β2 (ng/ml) | 0.003-44.788 | 1.1 ± 1.0 | 1.5 ± 1.1 | 1.2 ± 0.7 | 1.1 ± 0.5 | P = 0.656 |
| TGF-β3 (ng/ml) | 0.001-23.568 | ND | ND | ND | ND | |

### C. Discussion

This study clearly confirmed that administration of a CD14 antagonist antibody following STEMI was cardioprotective. This was observed in both the two dose and three dose groups. Important observations from the study include:
The isotype control had no effect on left ventricular systolic function at D7 vs saline control;
The third dose of the anti-CD14 Ab had no additional effect compared to the two dose anti-CD14 Ab protocol in any assessment;
The two dose anti-CD14 Ab treatment led to improved left ventricular systolic function (area change and ejection fraction [%]) at D7 compared to isotype and saline controls;
The three dose anti-CD14 Ab treatment led to similar improvements to two dose anti-CD14 Ab in left ventricular systolic function (area change and ejection fraction [%]) at D7 compared to isotype control;
Both the two and three dose protocols improved stroke volume and cardiac output at D7 compared to both controls;
Both the two and three dose protocols reduced lesion size (%), fibrosis and dV/dt minimum (left ventricular ejection rate), and increased stroke work (area of pressure-volume loop) at D7 compared to saline control; and
The two dose anti-CD14 Ab reduced myocardial CD68+ cell infiltrates at D7 compared to both controls.

The disclosure of every reference, patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1-20. (canceled)

21. A method for treating myocardial infarction (MI) in a subject, comprising administering an effective amount of a CD14 antagonist antibody to the subject.

22. The method of claim 21, wherein the CD14 antagonist antibody is administered to the subject up to 72 hours post-MI.

23. The method of claim 21, wherein the CD14 antagonist antibody is administered to the subject up to 12, 18, 24, 36 or 48 hours post-MI.

24. The method of claim 21, wherein the CD14 antagonist antibody is administered to the subject in 1, 2, 3 or more doses.

25. The method of claim 21, wherein the CD14 antagonist antibody is administered systemically.

26. The method of claim 21, wherein the MI is ST-segment elevation MI (STEMI).

27. The method of claim 21, wherein the MI is non-ST-segment elevation MI (NSTEMI).

28. The method of claim 21, wherein the CD14 antagonist antibody is selected from the group consisting of:
(i) an antibody that comprises: a) an antibody VL domain, or antigen binding fragment thereof, comprising L-CDR1, L-CDR2 and L-CDR3, wherein: L-CDR1 comprises the sequence RASESVDSFGNSFMH [SEQ ID NO: 7](3C10 L-CDR1); L-CDR2 comprises the sequence RAANLES [SEQ ID NO: 8](3C10 L-CDR2); and L-CDR3 comprises the sequence QQSYEDPWT [SEQ ID NO: 9](3C10 L-CDR3); and b) an antibody VH domain, or antigen binding fragment thereof, comprising H-CDR1, H-CDR2 and H-CDR3, wherein: H-CDR1 comprises the sequence SYAMS [SEQ ID NO: 10](3C10 H-CDR1); H-CDR2 comprises the application Ser. No. 18/024,738 Docket No.: 22975-20098.00 sequence SISSGGTTYYPDNVKG [SEQ ID NO: 11](3C10 H-CDR2); and H-CDR3 comprises the sequence GYYDYHY [SEQ ID NO: 12](3C10 H-CDR3);
(ii) an antibody that comprises: a) an antibody VL domain, or antigen binding fragment thereof, comprising L-CDR1, L-CDR2 and L-CDR3, wherein: L-CDR1 comprises the sequence RASESVDSYVNSFLH [SEQ ID NO: 13](28C5 L-CDR1); L-CDR2 comprises the sequence RASNLQS [SEQ ID NO: 14](28C5 L-CDR2); and L-CDR3 comprises the sequence QQSNEDPTT [SEQ ID NO: 15](28C5 L-CDR3); and b) an antibody VH domain, or antigen binding fragment thereof, comprising H-CDR1, H-CDR2 and H-CDR3, wherein: H-CDR1 comprises the sequence SDSAWN [SEQ ID NO: 16](28C5 H-CDR1); H-CDR2 comprises the sequence YISYSGSTSYNPSLKS [SEQ ID NO: 17](28C5 H-CDR2); and H-CDR3 comprises the sequence GLRFAY [SEQ ID NO: 18](28C5 H-CDR3);
(iii) an antibody that comprises: a) an antibody VL domain, or antigen binding fragment thereof, comprising L-CDR1, L-CDR2 and L-CDR3, wherein: L-CDR1 comprises the sequence RASESVDSYVNSFLH [SEQ ID NO: 13](IC14 L-CDR1); L-CDR2 comprises the sequence RASNLQS [SEQ ID NO: 14](IC14 L-CDR2); and L-CDR3 comprises the sequence QQSNEDPYT [SEQ ID NO: 27](IC14 L-CDR3); and b) an antibody VH domain, or antigen binding fragment thereof, comprising H-CDR1, H-CDR2 and H-CDR3, wherein: H-CDR1 comprises the sequence SDSAWN [SEQ ID NO: 16](IC14 H-CDR1); H-CDR2 comprises the sequence YISYSGSTSYNPSLKS [SEQ ID NO: 17](IC14 H-CDR2); and H-CDR3 comprises the sequence GLRFAY [SEQ ID NO: 18](IC14 H-CDR3); and
(iv) an antibody that comprises: a) an antibody VL domain, or antigen binding fragment thereof, comprising L-CDR1, L-CDR2 and L-CDR3, wherein: L-CDR1 comprises the sequence RASQDIKNYLN [SEQ ID NO: 19](18E12 L-CDR1); L-CDR2 comprises the sequence YTSRLHS [SEQ ID NO: 20](18E12 L-CDR2); and L-CDR3 comprises the sequence QRGDTLPWT [SEQ ID NO: 21](18E12 L-CDR3); and b) an antibody VH domain, or antigen binding fragment thereof, comprising H-CDR1, H-CDR2 and H-CDR3, wherein: H-CDR1 comprises the sequence NYDIS [SEQ ID NO: 22](18E12 H-CDR1); H-CDR2 comprises the sequence VIWTSGGTNYNSAFMS [SEQ ID NO: 23](18E12 H-CDR2); and H-CDR3 comprises the sequence GDGNFYLYNFDY [SEQ ID NO: 24](18E12 H-CDR3).

29. The method of claim 21, wherein the CD14 antagonist antibody is selected from the group consisting of:
(i) an antibody comprising:
a VL domain that comprises, consists or consists essentially of the sequence:
[SEQ ID NO: 1]
QSPASLAVSLGQRATISCRASESVDSFGNSFMHWYQQKAGQPPKS
SIYRAANLESGIPARFSGSGSRTDFTLTINPVEADDVATYFCQQS
YEDPWTFGGGTKLGNQ
(3C10 VL);
and
a VH domain that comprises, consists or consists essentially of the sequence:
[SEQ ID NO: 2]
LVKPGGSLKLSCVASGFTFSSYAMSWVRQTPEKRLEWVASISSGG
TTYYPDNVKGRFTISRDNARNILYLQMSSLRSEDTAMYYCARGYY
DYHYWGQGTTLTVSS
(3C10 VH);
(ii) an antibody comprising:
a VL domain that comprises, consists or consists essentially of the sequence:
[SEQ ID NO: 3]
QSPASLAVSLGQRATISCRASESVDSYVNSFLHWYQQKPGQPPKL
LIYRASNLQSGIPARFSGSGSRTDFTLTINPVEADDVATYCCQQS
NEDPTTFGGGTKLEIK
(28C5 VL);
and
a VH domain that comprises, consists or consists essentially of the sequence:
[SEQ ID NO: 4]
LQQSGPGLVKPSQSLSLTCTVTGYSITSDSAWNWIRQFPGNRLEW
MGYISYSGSTSYNPSLKSRISITRDTSKNQFFLQLNSVTTEDTAT
YYCVRGLRFAYWGQGTLVTVSA
(28C5 VH);
(iii) an antibody comprising:
a VL domain that comprises, consists or consists essentially of the sequence:
[SEQ ID NO: 25]
QSPASLAVSLGQRATISCRASESVDSYVNSFLHWYQQKPGQPPKL
LIYRASNLQSGIPARFSGSGSRTDFTLTINPVEADDVATYYCQQS
NEDPYTFGGGTKLEIK
(IC14 VL);
and
a VH domain that comprises, consists or consists essentially of the sequence:
[SEQ ID NO: 26]
LQQSGPGLVKPSQSLSLTCTVTGYSITSDSAWNWIRQFPGNRLEW
MGYISYSGSTSYNPSLKSRISITRDTSKNQFFLQLNSVTTEDTAT
YYCVRGLRFAYWGQGTLVTVSS
(IC14 VH);
and
(iv) an antibody comprising:
a VL domain that comprises, consists or consists essentially of the sequence:
[SEQ ID NO: 5]
QTPSSLSASLGDRVTISCRASQDIKNYLNWYQQPGGTVKVLIYYT
SRLHSGVPSRFSGSGSGTDYSLTISNLEQEDFATYFCQRGDTLPW
TFGGGTKLEIK
(18E12 VL);
and
a VH domain that comprises, consists or consists essentially of the sequence:
[SEQ ID NO: 6]
LESGPGLVAPSQSLSITCTVSGFSLTNYDISWIRQPPGKGLEWLG
VIWTSGGTNYNSAFMSRLSITKDNSESQVFLKMNGLQTDDTGIYY
CVRGDGNFYLYNFDYWGQGTTLTVSS
(18E12 VH).

30. The method of claim 21, wherein the CD14 antagonist antibody is humanized or chimeric.

31. The method of claim 21, wherein the CD14 antagonist antibody comprises:
a light chain comprising the amino acid sequence
[SEQ ID NO: 32]
DIVLTQSPASLAVSLGQRATISCRASESVDSYVNSFLHWYQQKPG
QPPKLLIYRASNLQSGIPARFSGSGSRTDFTLTINPVEADDVATY
YCQQSNEDPYTFGGGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC;
and
a heavy chain comprising the amino acid sequence:
[SEQ ID NO: 33]
DVQLQQSGPGLVKPSQSLSLTCTVTGYSITSDSAWNWIRQFPGNR
LEWMGYISYSGSTSYNPSLKSRISITRDTSKNQFFLQLNSVTTED
TATYYCVRGLRFAYWGQGTLVTVSSASTKGPSVFPLAPCSRSTSE
STAALGCLVKDYFPEPVTVSWNSGALTSGVHTFPAVLQSSGLYSL
SSVVTVPSSSLGTKTYTCNVDHKPSNTKVDKRVESKYGPPCPSCP
APEFLGGPSVFLFPPKPKDTLMISRTPEVTCVVVDVSQEDPEVQF
NWYVDGVEVHNAKTKPREEQFNSTYRVVSVLTVLHQDWLNGKEYK
CKVSNKGLPSSIEKTISKAKGQPREPQVYTLPPSQEEMTKNQVSL
TCLVKGFYPSDIAVEWESNGQPENNYKTTPPVLDSDGSFFLYSRL
TVDKSRWQEGNVFSCSVMHEALHNHYTQKSLSLSLGK.

32. The method of claim 21, wherein the CD14 antagonist antibody is administered in combination with an ancillary agent.

33. The method of claim 32, wherein the CD14 antagonist antibody and the ancillary agent are administered simultaneously or sequentially.

34. The method of claim 32, wherein the ancillary agent is selected from the group consisting of a fibrinolytic agent, a beta blocker, a high intensity statin, an angiotensin converting enzyme (ACE) inhibitor and a platelet inhibitor.

35. The method of claim 34, wherein the fibrinolytic agent is selected from the group consisting of streptokinase, anistreplase and a tissue plasminogen activator.

36. The method of claim 34, wherein the beta blocker is selected from the group consisting of acebutolol, atenolol, bisoprolol, metoprolol, nadolol, nebivolol and propranolol.

37. The method of claim 34, wherein the platelet inhibitor is selected from the group consisting of aspirin, a P2Y12 inhibitors and glycoprotein IIb/IIIa receptor antagonists.

38. The method of claim 21, wherein percutaneous coronary intervention (PCI) is performed on the subject.

39. The method of claim 38, wherein the CD14 antagonist antibody is administered within 72 hours of PCI.

40. The method of claim 38, wherein the PCI is angioplasty or stent placement.
